(19) United States
(12) Patent Application Publication
(10) Pub. No.: US 2010/0256014 A1
(43) Pub. Date: Oct. 7, 2010

(54) NOVEL AS160-LIKE PROTEIN, TEST SYSTEMS, METHODS AND USES INVOLVING IT FOR THE IDENTIFICATION OF DIABETES TYPE 2 THERAPEUTICS

(75) Inventors: Norbert Tennagels, Siegburg (DE); Daniela Baus, Frankfurt (DE); Kathrin Heermeier, Hochheim (DE); Werner Dittrich, Frankfurt (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/667,772

(22) PCT Filed: Jul. 23, 2008
(86) PCT No.: PCT/EP08/06024

§ 371 Date: Jun. 10, 2010

(30) Foreign Application Priority Data
Aug. 1, 2007 (EP) 07015086.7

(51) Int. Cl. C12Q 1/54; C40B 30/06; C12Q 1/68; C07K 14/435; C07H 21/04; C07K 16/18; C12N 5/10

(52) U.S. Cl. 506/10; 435/14; 435/6; 530/350; 536/23.5; 530/387.9; 435/325; 536/24.5

(57) ABSTRACT

The present invention relates to novel AKT substrate 160 kDa-like protein (AS160-like protein), to a method of identifying a substance altering glucose uptake and/or GLUT4 translocation to the plasma membrane of a cell comprising contacting a test system comprising AKT substrate 160 kDa-like protein (AS160-like protein) with a test substance, and identifying a test substance as a substance altering glucose uptake of a cell by detecting a signal indicative for altered glucose uptake of a cell; a test system comprising a gene coding for the AKT substrate 160 kDa-like protein (AS 160-like protein) and an inducible promoter providing for controllable expression of the gene; the use of the test system for the identification of a substance improving glucose uptake and/or GLUT4 translocation to the plasma membrane of a cell; and the use of AS160-like protein in a model for type 2 diabetes.

The present invention relates to novel AKT substrate 160 kDA-like protein (AS 160-like protein); a method of identifying a substance altering glucose uptake of a cell comprising contacting a test system comprising novel AKT substrate 160 kDa-like protein (AS160-like protein) with a test substance, and identifying a test substance as a substance altering glucose uptake of a cell by detecting a signal indicative for altered glucose uptake of a cell; a test system comprising a gene coding for the AKT substrate 160 kDa-like protein (AS160-like protein) and an inducible promoter providing for controllable expression of the gene; the use of the test system for the identification of a substance improving glucose uptake into a cell; and the use of AS160-like protein in a model for type 2 diabetes.

Diabetes mellitus is a metabolic disorder characterized by hyperglycemia and other signs, as distinct from a single illness or condition. The World Health Organization recognizes three main forms of diabetes: type 1, type 2, and gestational diabetes (occurring during pregnancy), which have similar signs, symptoms, and consequences, but different causes and population distributions. Ultimately, all forms are due to the beta cells of the pancreas being unable to produce sufficient insulin to prevent hyperglycemia.

Type 1 is usually due to autoimmune destruction of the pancreatic beta cells which produce insulin. Type 2 is characterized by tissue-wide insulin resistance, particularly of insulin-sensitive tissues comprising adipose tissue, liver and skeletal muscle, and varies widely; it sometimes progresses to loss of beta cell function. Gestational diabetes is similar to type 2-diabetes, in that it involves insulin resistance caused by hormones of pregnancy.

Types 1 and 2 are incurable chronic conditions, but have been treatable and are usually managed with a combination of dietary treatment and medicaments including insulin supplementation.

Diabetes can cause many complications such as hypoglycemia, ketoacidosis or nonketotic hyperosmolar coma. Serious long-term complications include cardiovascular disease (doubled risk), chronic renal failure (diabetic nephropathy is the main cause of dialysis in developed world adults), retinal damage (which can lead to blindness and is the most significant cause of adult blindness in the non-elderly in the developed world), nerve damage (of several kinds), and microvascular damage, which may cause erectile dysfunction (impotence) and poor healing. Poor healing of wounds, particularly of the feet, can lead to gangrene which can require amputation—the leading cause of non-traumatic amputation in adults in the developed world.

Because insulin is the principal hormone that regulates uptake of glucose into most cells from the blood (primarily muscle and adipocytes), deficiency of insulin or the insensitivity of its receptors plays a central role in all forms of diabetes mellitus. Insulin is released into the blood by β-cells in the pancreas in response to rising levels of blood glucose (e.g., after a meal). Insulin enables most body cells (about ⅔ is the usual estimate, including muscle cells and adipose tissue) to absorb glucose from the blood.

Type 2 diabetes mellitus is due to a combination of defective insulin secretion and insulin resistance or reduced insulin sensitivity of insulin-sensitive tissues, particularly adipose tissue, liver and skeletal muscle. In the early stage the predominant abnormality is reduced insulin sensitivity, characterized by elevated levels of insulin in the blood. At this stage, hyperglycemia can be reversed by a variety of measures and medications that improve insulin sensitivity or reduce glucose production by the liver, but as the disease progresses, the impairment of insulin secretion worsens, and therapeutic replacement of insulin often becomes necessary.

Usually, type 2 diabetes is first treated by attempts to change physical activity, the diet (generally to decrease carbohydrate intake), and weight loss. The usual next step, if necessary, is treatment with oral antidiabetic drugs. As insulin production is initially only moderately impaired in type 2 diabetics, oral medication can still be used to improve insulin production, to regulate inappropriate release of glucose by the liver and to substantially attenuate insulin resistance.

Adequate treatment of diabetes in early the stage, particularly improvement insulin sensitivity of adipose tissue, liver and skeletal muscle, may protract, retard and/or prevent progression of the disease.

Accordingly, a first object of the invention was to better understand the molecular background involved in the glucose metabolism, thus helping to better search for new potential drugs improving insulin sensitivity of cells, particularly of cells of skeletal muscle, adipose tissue and/or liver, which are the main insulin-sensitive tissues.

Surprisingly, two novel isoforms (isoforms 2 and 3) of AKT substrate 160 kDa (AS160, also referred to as Tbc1 D4 or AS160, isoform 1) have now been identified by the inventors. In the following, the term “AS160-like protein”, refers to either or both novel isoforms of AS160 i.e. to isoforms 2 and/or 3. According to one embodiment, the term “AS160-like protein” refers to both isoforms, according to another, it refers to isoforms 2 and according to yet another embodiment, it refers to isoform 3. According to a preferred embodiment, the term “AS 160-like protein” refers to isoforms 2. AS160, isoforms AS160, isoform 2 is expressed in the six main insulin-sensitive tissues, i.e. adipose tissue, liver, skeletal muscle, heart, brain and pancreatic tissue (see FIG. 2 A, B). In contrast to this, the isoform 1 of AS160, is predominantly detected in skeletal muscle and heart. The third isoform (AS160, isoform 3), lacking exon 12 of AS160 (FIG. 1) is also mainly expressed in heart and skeletal muscle. (FIG. 2C). As an ineffective insulin action is a hallmark of type 2 diabetes, AS160-like protein provides a novel target to study the molecular basis of insulin-resistance in insulin-sensitive tissues. Additionally, AS160-like protein may be used to identify substances which could improve glucose uptake, and therefore insulin sensitivity, in the relevant tissues.

Components of the intracellular signal transduction pathway involving AS160-like protein were identified which allows for studying interaction of a substance with the respective signal transduction pathway at different levels. It could be shown that insulin-stimulated signal transduction relating to AS160-like protein involves phosphorylation of AS160 and AKT, involvement of PI3K (PI3-kinase) and MEKK/ERK kinases. Unexpectedly, the inventors found that overexpression of AS160-like results in enhanced translocation of GLUT4 to the plasma membrane and increase in glucose uptake.

They also found that a test system involving AS160-like protein may be used to study glucose uptake of cells under high glucose conditions. This may be particularly important for a diabetes model or for the identification of a suitable therapeutic for the treatment and/or prevention of diabetes, as this disease is characterized by increased levels of glucose in the blood. Accordingly, testing a substance capable of altering, particularly improving, glucose uptake under this condition (high glucose) might be beneficial for the identification of a new therapeutic.

Accordingly, AS160-like protein may be used in a method of identifying a substance altering, particularly increasing, glucose uptake of a cell and, therefore, having a potential for the treatment or prevention of type 2 diabetes.

Therefore, the present invention provides in a first aspect a method of identifying a substance altering glucose uptake and/or GLUT4 translocation of a cell comprising

(a) contacting a test system comprising AKT substrate 160kDa-like protein (AS160-like protein) with a test substance, and

(b) identifying a test substance as a substance altering glucose uptake and/or GLUT4 translocation of a cell by detecting a signal indicative for altered glucose uptake of a cell.

“Altering glucose uptake of a cell” in the context of the present invention means a change, either increase or decrease, of glucose uptake of a cell. Preferably the glucose uptake of a cell is increased.

In the context of the present invention, the glucose uptake of a cell is altered, i.e. decreased or increased in comparison to a control, if the glucose uptake of a cell contacted with the (test) substance is significantly lower or higher, respectively, than that of the control (e.g. the same cell not contacted with the (test) substance). The person skilled in the art knows statistical procedures to assess whether two values are significantly different from each other such as Student's t-test or chi-squared test (see also Examples for suitable test methods).

In a preferred embodiment of increased glucose uptake, the glucose uptake of a cell amounts to at least 110%, preferably to at least 125%, more preferably to at least 150%, 160%, 170%, 180% or 190%, still more preferably to at least 200% and most preferably to at least 300% of the control.

As detailed above, for the treatment or prevention of type 2 diabetes it is particularly important to modify glucose uptake of an insulin-sensitive tissue. Accordingly, it is preferred that the method of the invention allows for the identification of a substance altering, preferably increasing, the glucose uptake in at least one, preferably at least two, more preferably at least three or at least four or at least five or at least six insulin-sensitive tissue(s). Examples of insulin-sensitive tissues include, without limitation, adipose tissue, liver, skeletal muscle, pancreatic tissue, myocardium, vascular smooth muscle and active mammary gland.

However, the six main insulin-sensitive tissues are adipose tissue, liver skeletal muscle, heart, brain and pancreatic tissue. Accordingly, the substance preferably alters, more preferably increases, glucose uptake in at least one, two, three, four, five or six or more of these tissues, i.e. adipose tissue, skeletal muscle, heart, brain, pancreatic tissue and/or liver.

If glucose uptake is altered, preferably increased, in one tissue, this could be, for example, any of: adipose tissue, liver, heart, brain, pancreatic tissue or skeletal muscle.

If glucose uptake is altered, preferably increased, in two tissues, this could be, for example,

adipose tissue and liver;

adipose tissue and skeletal muscle; or

liver and skeletal muscle, or any other combination of two of the six main insulin-sensitive tissues as listed above.

If glucose uptake is altered, preferably increased, in three tissues, this could be, for example, adipose tissue, liver and skeletal muscle or any other combination of three of the above-listed six main insulin-sensitive tissues.

If glucose uptake is altered, preferably increased in four tissues, this could be, for example, adipose tissue, liver, skeletal muscle and brain or any other combination of four of the above-listed six main insulin-sensitive tissues.

If glucose uptake is altered, preferably increased in five tissues, this could be, for example, adipose tissue, liver, skeletal muscle, brain and heart, or any other combination of five of the above-listed six main insulin-sensitive tissues.

The main cell types present in adipose tissue, liver, heart, brain, pancreatic tissue and skeletal muscle are adipocytes, hepatocytes, heart muscle cells, neuronal cells pancreatic cells, beta cells and skeletal muscle cells, respectively. Accordingly, the substance preferably alters, more preferably increases, glucose uptake in at least one, two or three of these cell types, i.e. adipocytes, hepatocytes and/or skeletal muscle cells.

If glucose uptake is altered, preferably increased, in one cell type, this could be, for example in adipocytes, hepatocytes, heart muscle cells, neuronal cells pancreatic cells, beta cells or skeletal muscle cells.

If glucose uptake is altered, preferably increased, in two cell types, this could be, for example,

adipocytes and hepatocytes;

adipocytes and skeletal muscle cells; or

hepatocytes and skeletal muscle cells or any other combination of the main cell types present in the six main insulin-sensitive tissues as listed above.

If glucose uptake is altered, preferably increased, in three cell types, this could be, for example, adipocytes, hepatocytes and skeletal muscle cells or any other combination of the above listed main cell types present in the six main insulin-sensitive tissues as listed above.

If glucose uptake is altered, preferably increased in four, five or more of the main cell types as listed above for the six main insulin-sensitive tissues, this can be any combination of four, five or more of those cell types.

As detailed above, the method of the invention involves a test system comprising AS160-like protein. According to one embodiment (isoform 2 of AS160), AS160-like protein is derived from AKT substrate 160 kDa (AS160), wherein in comparison to AS160 the sequence encoded by exons 11 and 12 in AS160 is missing in AS160-like protein (see also FIG. 1). According to another embodiment (isoform3 of AS160), AS160-like protein is derived from AKT substrate 160 kDa (AS160), wherein in comparison to AS160, the sequence lacks exon 12.

Additionally, further mutations may be present as detailed below.

AS160 (AKT substrate 160 kDa; NM_{—}014832 (EMBL)) was originally identified as a substrate of the protein kinase AKT in 3T3 adipocytes (Kane et al., 2002). Additional studies demonstrated that AS160 also plays a role in skeletal muscles of mice, rats and humans. Insulin, contraction or AICAR (5-aminoimidozole-4-carboxamide 1β-D-ribonucleoside, cAMP-dependent protein kinase (cAMPK) activator) increase phosphorylation of AS160 on two sites (Ser 588 and Thr 642) which lie in characteristic motifs predicted for AKT phosphorylation (RXRXXS/T) (Kane et al., supra). A prominent feature of AS160 is the presence of a GTPase activating domain for Rab proteins. These small G-proteins are required for membrane trafficking. In this context, recent data provide evidence that AS160 links signals downstream of AKT with the insulin-stimulated translocation of GLUT4 (Sano et al., 2003). AS160 activation is reduced in patients with type II diabetes, resulting in an impaired GLUT4 translocation (Karisson et al., 2005b). Overexpression of full-length AS160 in adipocytes did not alter the basal or insulin-stimulated surface-to-total distribution of GLUT4 indicating that the amount of AS160 seems not to be rate-limiting (Zeigerer et al., 2004; Sano et al., 2003). Experiments with mutant AS160 (containing 4 mutated phosphorylation sites) showed that GLUT4 translocation is markedly reduced (Sano et al., 2003). Additionally, a functional GAP (GTPase-activating protein) domain of AS160 is required for GLUT4 translocation.

AS 160, isoforms 2: In the gene encoding novel isoform 2 of AS160, exons 11 and 12 are missing in comparison to the full-length AS160 gene (see Examples 1 and 2). In case of the human gene, the nucleotides encoding amino acids 678 to 740 are missing in comparison to the human full-length AS160 as defined by the sequence NM_{—}014832 (see EMBL data base). Furthermore, two mismatches were identified at positions nt 606 (silent) and nt 3827 (Ala→Val). In addition, the clone of Example 2 contained a 3 by deletion (nt 2594-2596) that was also found in human placenta cDNA but not in human brain cDNA. For the expression clone of the isoform lacking exons 11 and 12, which was used in the Examples, the deleted 3 bp sequence was reintroduced to resemble more closely the full length sequence of NM_{—}014832 (EMBL). The resulting DNA sequence encoding isoform 2 of AS160 (SEQ ID NO: 1) is given in the following:

DNA Sequence encoding isoform 2 of AS160 (SEQ ID NO: 1)

ATGGAGCCGCCCAGCTGCATTCAGGATGAGCCGTTCCCGCACCCCCTGGA
GCCCGAGCCGGGCGTCTCAGCTCAGCCCGGCCCCGGGAAGCCAAGCGATA
AGCGGTTCCGGCTGTGGTACGTTGGGGGGTCGTGCCTGGACCACAGGACC
ACGCTGCCTATGCTGCCCTGGCTCATGGCCGAGATCCGCAGGCGCAGCCA
GAAGCCCGAGGCGGGCGGCTGCGGGGCGCCGGCGGCCCGAGAGGTGATCC
TGGTGCTCAGCGCGCCCTTCCTGCGTTGCGTCCCCGCGCCGGGCGCTGGG
GCCTCGGGGGGCACTAGTCCGTCGGCCACGCAGCCCAACCCGGCGGTATT
CATCTTCGAGCACAAGGCGCAGCATATCTCGCGCTTCATCCACAACAGCC
ACGACCTCACCTACTTTGCCTACCTGATCAAGGCGCAGCCCGACGACCCC
GAGTCGCAGATGGCCTGCCACGTTTTCCGCGCCACAGACCCCAGCCAGGT
TCCTGATGTTATTAGCAGCATAAGGCAATTATCTAAAGCGGCCATGAAAG
AGGATGCCAAACCCAGCAAAGATAATGAGGACGCCTTTTACAACTCTCAG
AAGTTTGAAGTCCTGTACTGTGGAAAGGTGACCGTGACCCACAAGAAGGC
CCCCTCAAGCCTCATCGATGACTGCATGGAGAAGTTCAGCCTGCACGAAC
AGCAGCGCCTGAAGATCCAAGGCGAGCAGCGCGGTCCGGACCCAGGAGAG
GACCTGGCTGACTTGGAGGTGGTGGTGCCCGGGTCCCCCGGAGACTGCCT
GCCGGAGGAGGCTGACGGCACCGACACCCACCTTGGCTTACCTGCCGGGG
CCAGCCAGCCTGCCCTGACCAGCTCTCGGGTCTGCTTCCCTGAGCGGATT
TTGGAAGATTCTGGCTTTGATGAGCAGCAGGAGTTTCGGTCTCGGTGCAG
CAGTGTCACCGGCGTGCAACGGAGAGTTCACGAGGGCAGCCAGAAATCCC
AGCCGCGACGGAGACACGCGAGCGCACCCAGTCACGTCCAGCCCTCGGAC
TCGGAGAAGAACAGGACCATGCTCTTCCAGGTTGGGCGATTTGAGATTAA
CCTTATCAGTCCAGACACTAAATCAGTTGTGCTAGAAAAGAATTTTAAAG
ATATCTCCTCTTGTTCTCAGGGTATAAAGCATGTGGATCACTTTGGCTTT
ATCTGCCGGGAGTCTCCAGAGCCTGGACTTAGCCAGTATATTTGTTATGT
ATTCCAGTGTGCCAGCGAATCTCTGGTTGATGAGGTAATGCTGACTCTGA
AACAGGCCTTCAGTACGGCGGCTGCCCTGCAGAGTGCCAAGACGCAGATT
AAACTGTGTGAGGCCTGCCCGATGCACTCTTTGCATAAGCTCTGTGAAAG
GATTGAAGGTCTCTACCCACCAAGAGCCAAGCTGGTGATACAGAGGCATC
TCTCATCACTGACAGATAATGAGCAAGCTGACATCTTTGAAAGAGTTCAG
AAAATGAAGCCAGTCAGTGACCAGGAAGAAAATGAACTTGTGATTTTACA
CCTGAGGCAGCTGTGTGAAGCCAAGCAGAAGACACACGTGCACATCGGGG
AAGGCCCTTCTACTATTTCAAATAGTACAATCCCAGAAAATGCAACAAGC
AGTGGAAGGTTCAAACTTGACATTCTGAAAAATAAAGCTAAGAGATCCTT
AACTAGCTCCCTGGAAAATATCTTCTCAAGGGGAGCTAACAGAATGAGAG
GTCGGCTTGGAAGTGTGGACAGTTTTGAACGGTCCAACAGTCTTGCTTCA
GAGAAGGACTACTCACCAGGGGATTCTCCACCAGGGACACCGCCAGCGTC
CCCACCGTCCTCAGCTTGGCAAACGTTTCCCGAAGAGGATTCCGACTCCC
CGCAGTTTCGAAGACGGGCACACACGTTCAGCCACCCACCTTCAAGCACA
AAGAGAAAGCTGAATTTGCAGGATGGGAGGGCTCAGGGTGTGCGTTCCCC
TCTGCTGAGGCAGAGCTCCAGTGAACAGTGCAGTGATGGAGAAGGGAGAA
AAAGGACCTCATCTACCTGCAGCAATGAGTCCCTAAGTGTGGGAGGAACC
TCTGTCACTCCTCGCCGGATCTCCTGGCGGCAGCGCATTTTCCTCAGGGT
TGCTTCTCCCATGAACAAATCTCCCTCAGCAATGCAACAGCAAGATGGAT
TGGACAGGAACGAGCTGCTGCCACTGTCCCCCCTCTCTCCAACCATGGAG
GAGGAACCGCTGGTTATATTCCTGTCTGGGGAGGATGACCCAGAAAAGAT
TGAAGAAAGAAAGAAATCAAAAGAACTGAGGAGCTTGTGGAGAAAAGCTA
TACACCAACAAATCTTGTTACTTCGAATGGAAAAAGAAAACCAGAAACTT
GAAGGAGCAAGCAGAGATGAACTCCAGTCCAGAAAAGTTAAATTAGACTA
TGAAGAAGTTGGTGCATGTCAGAAAGAGGTCTTAATAACTTGGGATAAGA
AGTTGTTAAACTGCAGAGCTAAAATCAGATGTGATATGGAAGATATTCAT
ACTCTTCTTAAAGAAGGAGTTCCCAAAAGTCGACGAGGAGAAATTTGGCA
GTTTCTGGCTTTACAGTACCGACTCAGACACAGATTGCCTAATAAACAAC
AGCCTCCTGACATATCCTATAAGGAACTTTTGAAGCAGCTCACTGCTCAG
CAGCATGCGATTCTCGTGGATTTAGGAAGGACGTTTCCTACTCACCCTTA
CTTTTCAGTACAGCTTGGGCCAGGACAGCTGTCACTGTTTAACCTCCTGA
AAGCCTATTCTTTGCTGGACAAAGAAGTGGGATACTGTCAGGGGATCAGC
TTTGTGGCTGGAGTCCTGCTTCTGCACATGAGTGAAGAGCAAGCCTTTGA
AATGCTGAAATTCCTCATGTATGACCTCGGCTTCCGCAAGCAGTACAGAC
CTGACATGATGTCGCTGCAGATTCAAATGTACCAGCTGTCCAGGCTCCTT
CATGACTATCACAGAGATCTCTACAATCACCTTGAAGAAAATGAAATCAG
CCCCAGTCTTTATGCTGCCCCCTGGTTCCTCACATTGTTTGCCTCTCAGT
TTTCATTAGGATTTGTAGCCAGAGTTTTTGATATTATTTTTCTTCAGGGA
ACTGAAGTTATATTCAAGGTTGCACTCAGCCTACTGAGCAGCCAAGAGAC
ACTTATAATGGAATGTGAGAGCTTTGAAAATATTGTTGAGTTTCTTAAAA
ACACGCTACCTGATATGAATACCTCTGAAATGGAAAAAATTATTACCCAG
GTTTTTGAGATGGATATTTCTAAGCAGTTGCATGCCTATGAGGTGGAATA
TCATGTGCTACAGGATGAGCTTCAGGAATCTTCATATTCCTGTGAGGATA
GTGAAACTTTGGAGAAGCTGGAGAGGGCCAATAGCCAACTGAAAAGACAA
AACATGGACCTCCTAGAAAAATTACAGGTAGCTCATACTAAAATCCAGGC
CTTGGAATCAAACCTGGAAAATCTTTTGACGAGAGAGACCAAAATGAAGT
CTTTAATCCGGACCCTGGAACAAGAAAAAATGGCTTATCAAAAGACAGTG
GAGCAACTCCGGAAGCTGCTGCCCGCGGATGCTCTAGTCAATTGTGACCT
GTTGCTGAGAGACCTAAACTGCAACCCTAACAACAAAGCCAAGATAGGAA
ATAAGCC

The translated amino acid sequence via EditSeq (Lasergene) (SEQ ID NO: 3) of novel isoform 2 of AS160 is given in the following:

MEPPSCIQDEPFPHPLEPEPGVSAQPGPGKPSDKRFRLWYVGGSCLDHRT
TLPMLPWLMAEIRRRSQKPEAGGCGAPAAREVILVLSAPFLRCVPAPGAG
ASGGTSPSATQPNPAVFIFEHKAQHISRFIHNSHDLTYFAYLIKAQPDDP
ESQMACHVFRATDPSQVPDVISSIRQLSKAAMKEDAKPSKDNEDAFYNSQ
KFEVLYCGKVTVTHKKAPSSLIDDCMEKFSLHEQQRLKIQGEQRGPDPGE
DLADLEVWPGSPGDCLPEEADGTDTHLGLPAGASQPALTSSRVCFPERIL
EDSGFDEQQEFRSRCSSVTGVQRRVHEGSQKSQPRRRHASAPSHVQPSDS
EKNRTMLFQVGRFEINLISPDTKSVVLEKNFKDISSCSQGIKHVDHFGFI
CRESPEPGLSQYICYVFQCASESLVDEVMLTLKQAFSTAAALQSAKTQIK
LCEACPMHSLHKLCERIEGLYPPRAKLVIQRHLSSLTDNEQADIFERVQK
MKPVSDQEENELVILHLRQLCEAKQKTHVHIGEGPSTISNSTIPENATSS
GRFKLDILKNKAKRSLTSSLENIFSRGANRMRGRLGSVDSFERSNSLASE
KDYSPGDSPPGTPPASPPSSAWQTFPEEDSDSPQFRRRAHTFSHPPSSTK
RKLNLQDGRAQGVRSPLLRQSSSEQCSDGEGRKRTSSTCSNESLSVGGTS
VTPRRISWRQRIFLRVASPMNKSPSAMQQQDGLDRNELLPLSPLSPTMEE
EPLVIFLSGEDDPEKIEERKKSKELRSLWRKAIHQQILLLRMEKENQKLE
GASRDELQSRKVKLDYEEVGACQKEVLITWDKKLLNCRAKIRCDMEDIHT
LLKEGVPKSRRGEIWQFLALQYRLRHRLPNKQQPPDISYKELLKQLTAQQ
HAILVDLGRTFPTHPYFSVQLGPGQLSLFNLLKAYSLLDKEVGYCQGISF
VAGVLLLHMSEEQAFEMLKFLMYDLGFRKQYRPDMMSLQIQMYQLSRLLH
DYHRDLYNHLEENEISPSLYAAPWFLTLFASQFSLGFVARVFDIIFLQGT
EVIFKVALSLLSSQETLIMECESFENIVEFLKNTLPDMNTSEMEKIITQV
FEMDISKQLHAYEVEYHVLQDELQESSYSCEDSETLEKLERANSQLKRQN
MDLLEKLQVAHTKIQALESNLENLLTRETKMKSLIRTLEQEKMAYQKTVE
QLRKLLPADALVNCDLLLRDLNCNPNNKAKIGNKP

As 160, isoform 3: In the gene encoding novel isoform 3 of AS160, exon 12 is deleted compared to full length AS160 gene. This exon corresponds to amino acids 733 to 740 with respect to NM_{—}014832 (see EMBL data base). The resulting DNA sequence encoding isoform 3 of AS160 (SEQ ID NO: 22) is given in the following:

DNA Sequence encoding isoform3 of AS160 (SEQ ID No: 22)

(SEQ ID NO: 22)

ATGGAGCCGCCCAGCTGCATTCAGGATGAGCCGTTCCCGCACCCCCTGGA
GCCCGAGCCGGGCGTCTCAGCTCAGCCCGGCCCCGGGAAGCCAAGCGATA
AGCGGTTCCGGCTGTGGTACGTTGGGGGGTCGTGCCTGGACCACAGGACC
ACGCTGCCTATGCTGCCCTGGCTCATGGCCGAGATCCGCAGGCGCAGCCA
GAAGCCCGAGGCGGGCGGCTGCGGGGCGCCGGCGGCCCGAGAGGTGATCC
TGGTGCTCAGCGCGCCCTTCCTGCGTTGCGTCCCCGCGCCGGGCGCTGGG
GCCTCGGGGGGCACTAGTCCGTCGGCCACGCAGCCCAACCCGGCGGTATT
CATCTTCGAGCACAAGGCGCAGCATATCTCGCGCTTCATCCACAACAGCC
ACGACCTCACCTACTTTGCCTACCTGATCAAGGCGCAGCCCGACGACCCC
GAGTCGCAGATGGCCTGCCACGTTTTCCGCGCCACAGACCCCAGCCAGGT
TCCTGATGTTATTAGCAGCATAAGGCAATTATCTAAAGCGGCCATGAAAG
AGGATGCCAAACCCAGCAAAGATAATGAGGACGCCTTTTACAACTCTCAG
AAGTTCGAAGTCCTGTACTGTGGAAAGGTGACCGTGACCCACAAGAAGGC
CCCCTCAAGCCTCATCGATGACTGCATGGAGAAGTTCAGCCTGCACGAAC
AGCAGCGCCTGAAGATCCAAGGGGAGCAGCGCGGTCCGGACCCAGGAGAG
GACCTGGCTGACTTGGAGGTGGTGGTGCCCGGGTCCCCCGGAGACTGCCT
GCCGGAGGAGGCTGACGGCACCGACACCCACCTTGGCTTACCTGCCGGGG
CCAGCCAGCCTGCCCTGACCAGCTCTCGGGTCTGCTTCCCTGAGCGGATT
TTGGAAGATTCTGGCTTTGATGAGCAGCAGGAGTTTCGGTCTCGGTGCAG
CAGTGTCACCGGCGTGCAACGGAGAGTTCACGAGGGCAGCCAGAAATCCC
AGCCGCGACGGAGACACGCGAGCGCACCCAGTCACGTCCAGCCCTCGGAC
TCGGAGAAGAACAGGACCATGCTCTTCCAGGTTGGGCGATTTGAGATTAA
CCTTATCAGTCCAGACACTAAATCAGTTGTGCTAGAAAAGAATTTTAAAG
ATATCTCCTCTTGTTCTCAGGGTATAAAGCATGTGGATCACTTTGGCTTT
ATCTGCCGGGAGTCTCCAGAGCCTGGACTTAGCCAGTATATTTGTTATGT
ATTCCAGTGTGCCAGCGAATCTCTGGTTGATGAGGTAATGCTGACTCTGA
AACAGGCCTTCAGTACGGCGGCTGCCCTGCAGAGTGCCAAGACGCAGATT
AAACTGTGTGAGGCCTGCCCGATGCACTCTTTGCATAAGCTCTGTGAAAG
GATTGAAGGTCTCTACCCACCAAGAGCCAAGCTGGTGATACAGAGGCATC
TCTCATCACTGACAGATAATGAGCAAGCTGACATCTTTGAAAGAGTTCAG
AAAATGAAGCCAGTCAGTGACCAGGAAGAAAATGAACTTGTGATTTTACA
CCTGAGGCAGCTGTGTGAAGCCAAGCAGAAGACACACGTGCACATCGGGG
AAGGCCCTTCTACTATTTCAAATAGTACAATCCCAGAAAATGCAACAAGC
AGTGGAAGGTTCAAACTTGACATTCTGAAAAATAAAGCTAAGAGATCCTT
AACTAGCTCCCTGGAAAATATCTTCTCAAGGGGAGCTAACAGAATGAGAG
GTCGGCTTGGAAGTGTGGACAGTTTTGAACGGTCCAACAGTCTTGCTTCA
GAGAAGGACTACTCACCAGGGGATTCTCCACCAGGGACACCGCCAGCGTC
CCCACCGTCCTCAGCTTGGCAAACGTTTCCCGAAGAGGATTCCGACTCCC
CGCAGTTTCGAAGACGGGCACACACGTTCAGCCACCCACCTTCAAGCACA
AAGAGAAAGCTGAATTTGCAGGATGGGAGGGCTCAGGGTGTGCGTTCCCC
TCTGCTGAGGCAGAGCTCCAGTGAACAGTGCAGCAATCTTTCGTCAGTTC
GACGCATGTACAAGGAGAGTAATTCTTCCTCCAGTCTTCCAAGTCTTCAC
ACTTCCTTCTCTGCCCCTTCCTTCACTGCCCCCTCTTTCCTGAAAAGCTT
TTACCAGAATTCAGGTAGACTGTCCCCACAGTATGAAAATGAAATCAGAC
TGATGGAGAAGGGAGAAAAAGGACCTCATCTACCTGCAGCAATGAGTCCC
TAAGTGTGGGAGGAACCTCTGTCACTCCTCGCCGGATCTCCTGGCGGCAG
CGCATTTTCCTCAGGGTTGCTTCTCCCATGAACAAATCTCCCTCAGCAAT
GCAACAGCAAGATGGATTGGACAGGAACGAGCTGCTGCCACTGTCCCCCC
TCTCTCCAACCATGGAGGAGGAACCGCTGGTTGTATTCCTGTCTGGGGAG
GATGACCCAGAAAAGATTGAAGAAAGAAAGAAATCAAAAGAACTGAGGAG
CTTGTGGAGAAAAGCTATACACCAACAAATCTTGTTACTTCGAATGGAAA
AAGAAAACCAGAAACTTGAAGCAAGCAGAGATGAACTCCAGTCCAGAAAA
GTTAAATTAGACTATGAAGAAGTTGGTGCATGTCAGAAAGAGGTCTTAAT
AACTTGGGATAAGAAGTTGTTAAACTGCAGAGCTAAAATCAGATGTGATA
TGGAAGATATTCATACTCTTCTTAAAGAAGGAGTTCCCAAAAGTCGACGA
GGAGAAATTTGGCAGTTTCTGGCTTTACAGTACCGACTCAGACACAGATT
GCCTAATAAACAACAGCCTCCTGACATATCCTATAAGGAACTTTTGAAGC
AGCTCACTGCTCAGCAGCATGCGATTCTCGTGGATTTAGGAAGGACGTTT
CCTACTCACCCTTACTTTTCAGTACAGCTTGGGCCAGGACAGCTGTCACT
GTTTAACCTCCTGAAAGCCTATTCTTTGCTGGACAAAGAAGTGGGATACT
GTCAGGGGATCAGCTTTGTGGCTGGAGTCCTGCTTCTGCACATGAGTGAA
GAGCAAGCCTTTGAAATGCTGAAATTCCTCATGTATGACCTCGGCTTCCG
CAAGCAGTACAGACCTGACATGATGTCGCTGCAGATTCAAATGTACCAGC
TGTCCAGGCTCCTTCATGACTATCACAGAGATCTCTACAATCACCTTGAA
GAAAATGAAATCAGCCCCAGTCTTTATGCTGCCCCCTGGTTCCTCACATT
GTTTGCCTCTCAGTTTTCATTAGGATTTGTAGCCAGAGTTTTTGATATTA
TTTTTCTTCAGGGAACTGAAGTTATATTCAAGGTTGCACTCAGCCTACTG
AGCAGCCAAGAGACACTTATAATGGAATGTGAGAGCTTTGAAAATATTGT
TGAGTTTCTTAAAAACACGCTACCTGATATGAATACCTCTGAAATGGAAA
AAATTATTACCCAGGTTTTTGAGATGGATATTTCTAAGCAGTTGCATGCC
TATGAGGTGGAATATCATGTGCTACAGGATGAGCTTCAGGAATCTTCATA
TTCCTGTGAGGATAGTGAAACTTTGGAGAAGCTGGAGAGGGCCAATAGCC
AACTGAAAAGACAAAACATGGACCTCCTAGAAAAATTACAGGTAGCTCAT
ACTAAAATCCAGGCCTTGGAATCAAACCTGGAAAATCTTTTGACGAGAGA
GACCAAAATGAAGTCTTTAATCCGGACCCTGGAACAAGAAAAAATGGCTT
ATCAAAAGACAGTGGAGCAACTCCGGAAGCTGCTGCCCGCGGATGCTCTA
GTCAATTGTGACCTGTTGCTGAGAGACCTAAACTGCAACCCTAACAACAA
AGCCAAGATAGGAAATAAGCCATAATTGAAG

The translated amino acid sequence via EditSeq (Lasergene) (SEQ ID NO:23) of novel isoform3 of AS160 is given in the following:

(SEQ ID NO: 23)

MEPPSCIQDEPFPHPLEPEPGVSAQPGPGKPSDKRFRLWYVGGSCLDHRT
TLPMLPWLMAEIRRRSQKPEAGGCGAPAAREVILVLSAPFLRCVPAPGAG
ASGGTSPSATQPNPAVFIFEHKAQHISRFIHNSHDLTYFAYLIKAQPDDP
ESQMACHVFRATDPSQVPDVISSIRQLSKAAMKEDAKPSKDNEDAFYNSQ
KFEVLYCGKVTVTHKKAPSSLIDDCMEKFSLHEQQRLKIQGEQRGPDPGE
DLADLEVWPGSPGDCLPEEADGTDTHLGLPAGASQPALTSSRVCFPERIL
EDSGFDEQQEFRSRCSSVTGVQRRVHEGSQKSQPRRRHASAPSHVQPSDS
EKNRTMLFQVGRFEINLISPDTKSVVLEKNFKDISSCSQGIKHVDHFGFI
CRESPEPGLSQYICYVFQCASESLVDEVMLTLKQAFSTAAALQSAKTQIK
LCEACPMHSLHKLCERIEGLYPPRAKLVIQRHLSSLTDNEQADIFERVQK
MKPVSDQEENELVILHLRQLCEAKQKTHVHIGEGPSTISNSTIPENATSS
GRFKLDILKNKAKRSLTSSLENIFSRGANRMRGRLGSVDSFERSNSLASE
KDYSPGDSPPGTPPASPPSSAWQTFPEEDSDSPQFRRRAHTFSHPPSSTK
RKLNLQDGRAQGVRSPLLRQSSSEQCSNLSSVRRMYKESNSSSSLPSLHT
SFSAPSFTAPSFLKSFYQNSGRLSPQYENEIRLMEKGEKGPHLPAAMSP.
VWEEPLSLLAGSPGGSAFSSGLLLP.TNLPQQCNSKMDWTGTSCCHCPPS
LQPWRRNRWLYSCLGRMTQKRLKKERNQKN.GACGEKLYTNKSCYFEWKK
KTRNLKQAEMNSSPEKLN.TMKKLVHVRKRS..LGIRSC.TAELKSDVIW
KIFILFLKKEFPKVDEEKFGSFWLYSTDSDTDCLINNSLLTYPIRNF.SS
SLLSSMRFSWI.EGRFLLTLTFQYSLGQDSCHCLTS.KPILCWTKKWDTV
RGSALWLESCFCT.VKSKPLKC.NSSCMTSASASSTDLT.CRCRFKCTSC
PGSFMTITEISTITLKKMKSAPVFMLPPGSSHCLPLSFH.DL.PEFLILF
FFRELKLYSRLHSAY.AAKRHL.WNVRALKILLSFLKTRYLI.IPLKWKK
LLPRFLRWIFLSSCMPMRWNIMCYRMSFRNLHIPVRIVKLWRSWRGPIAN
.KDKTWTS.KNYR.LILKSRPWNQTWKIF.RERPK.SL.SGPWNKKKWLI
KRQWSNSGSCCPRML.SIVTCC.ET.TATLTTKPR.EISHN.

The term “AS 160-like protein” refers to both or either of isoforms 2 or 3 of AS160. The term “isoform 2 of AS160” refers to a protein, whose gene is a naturally occurring variant of the AS160 gene in which exons 11 and 12 are deleted. The term “isoforms 3 of AS160” refers to a protein, whose gene is a naturally occurring variant of the AS160 gene, in which exon 12 is lacking in comparison to the full-length AS160.

Additionally, short mismatches may be present in the AS 160-like protein, if the sequence of AS160-like protein is compared to that of AS160. A short mismatch is intended to relate to an addition, deletion, or substitution of up to 5 adjacent amino acids, preferably up to 4, more preferably up to 3, still more preferably up to 2, most preferably up to 1 adjacent amino acid. Within the naturally occurring AS160-like protein there may by up to 5 additions, deletions, and/or substitutions, preferably up to 4, more preferably up to 3, 2 or 1 additions, deletions, and/or substitutions in comparison to AS160.

Exemplary deletions and substitutions are those mentioned above, namely a deletion of 1 amino acid at a position corresponding to that encoded by nt 2594-2596 of the human AS160 gene or a substitution (e.g. Ala→Val) at a position corresponding to that encoded by nt 3827-3829 of the human AS160 gene. It is noted that it is intend that the AS160-like protein or the nucleic acid coding the same may also be derived from species other than human including, but not limited to mammal, such as monkey, rodent (e.g. mouse or rat), dog, cat, cattle, pig, horse, sheep, goat or to avian, such as chicken or to amphibian, such as frog; however, the mammalian or human amino acid and nucleic acid sequences are preferred. The positions in AS160 or AS160-like protein sequences of species other than human corresponding to positions of the human sequences specified herein may be determined by sequence alignments as known to the skilled practitioner.

In one embodiment of the invention AS160-like protein comprises or consists of the sequence of a naturally occurring AS160-like protein (such as SEQ ID NO: 3 or SEQ Id NO: 23) and C- and/or N-terminal additions, such as short C- and/or N-terminal sequences of at most 30, 25, 20, 15, 10, 9, 8, 7, 6, 5, 4, 3, 2, or 1 amino acids heterologous to the protein as defined below.

Accordingly, the feature “AS160-like protein” relates to, e.g.:

1) a protein encoded by a nucleic acid comprising or having 90% or more, preferably 95% or more, more preferably 97% or more, more preferably 99% or more sequence homology with a nucleic acid sequence according to SEQ ID NO:1 or SEQ ID NO: 22

2) a protein encoded by a nucleic acid comprising or having the nucleic acid sequence according to SEQ ID NO:1 or SEQ ID NO: 22, or

3) a protein encoded by a nucleic acid hybridizing with a nucleic acid having the nucleic acid sequence according to SEQ ID NO:1 or SEQ ID NO: 22 under conditions of stringency, or

4) a protein having the amino acid sequence according to SEQ ID NO:3 or SEQ ID NO: 23, or

5) a protein having an amino acid sequence of 95% or more, preferably 97% or more, more preferably 98% or more, more preferably 99% or more and preferably 99,5% or more sequence homology with SEQ ID NO:3 or SEQ ID NO: 23,

6) a protein having an amino acid sequence of known AS160 (preferably, of human AS 160 and more preferably of AS 160 according to the sequence NM_{—}014832 (see EMBL data base) but lacking amino acids 600 to 800, preferably lacking the amino acids 650 to 770, more preferably lacking the amino acids 670 to 750, more preferably the amino acids 675 to 745 and most preferably lacking the amino acids 678 to 740 when compared to this AS 160 amino acid sequence, or

7) a protein having an amino acid sequence of known AS160 (preferably, of human AS 160 and more preferably of AS 160 according to the sequence NM_{—}014832 (see EMBL data base) but lacking the amino acids encoded by exon 12, preferably lacking amino acids 733 to 740.

8) a functional fragment or a functional derivative of one of the AS 160-like proteins as defined above under 1 to 7,

the above proteins preferably having at least one of the functional characteristics of AS 160-like protein as specified above and below.

A fragment is a protein that carries one or more end-terminal (n- and/or c-terminal) or internal deletions of one, two or more amino acids, when compared to the full-length protein. A functional fragment of a protein is any fragment of this protein having at least one and preferably two or more of the functional characteristics of the full-length protein.

The term derivative of a protein comprises any type of modification of the protein in comparison to the naturally-occurring form (in the context of present application especially in comparison to AS 160-like according to SEQ ID NO:3 or SEQ ID NO:23), that is not a deletion. A functional derivative of a protein is any derivative of this protein having at least one and preferably two or more of the functional characteristics of the unmodified protein.

Present invention also comprises functional derivatives of fragments of AS160-like protein.

The determination of homology of amino acid or nucleic acid sequences can e.g. be made by use of the program GAP (GCG Program Package, Genetic Computer Group 1991) or any other of the programs known in the art.

Isolated polynucleotides and oligonucleotides can be used for hybridizing at different conditions of stringency.

A nucleic acid molecule can hybridise to another nucleic acid molecule when the single stranded forms of both molecules can anneal under suitable reaction (“annealing” or hybridisation) conditions (depending on temperature and ionic strength of the surrounding medium) to form a new double stranded nucleic acid molecule. Hybridisation requires that the two annealing nucleic acid molecules comprise complementary sequences. Depending on the selected annealing conditions, the stringency conditions, mismatches between the bases are possible without preventing double strand formation.

The term stringency describes reaction conditions that influence the specificity of hybridisation or annealing of two single stranded nucleic acid molecules. Stringency, and thus specificity of a reaction depends, inter alia, of the temperature and buffer-conditions used for a reaction: Stringency, and thus specificity, can e.g. be increased by increasing the reaction temperature and/or lowering the ion strength of the reaction-buffer. Suitable conditions of stringency for the hybridisation of nucleic acids depend on the length, the type of nucleic acid and their degree of complementarity. The variables are known in the state of the art. The greater the degree of similarity or homology between two annealing nucleotide sequences, the greater the melting temperature for hybridisation products of nucleic acids with those sequences. The relative stability of nucleic acid hybridisation is dependent according to the type of the single stranded nucleic acids forming the double strand:

RNA:RNA>DNA:RNA>DNA:DNA. For hybridisation products of greater than 100 nucleotides in length, equations for calculating the melting temperature are known in the art. For shorter hybridisation products (e.g. oligonucleotides) the calculation of the melting temperature is dependent on the length, wherein mismatches become more important.

Conditions of low stringency (and thus low reaction and hybridisation specificity) exist for example, if a hybridisation is performed at room temperature in 2×SSC-solution. Conditions of high stringency comprise e.g. a hybridisation reaction at 68° C. in 0.1×SSC and 0.1% SDS solution.

In the context of present invention the term “hybridising under conditions of stringency” refers to conditions for the performance of the hybridisation reaction and the following washing procedure, at which nucleotide sequences with a certain complementarity typically remain hybridised. The choice of such conditions for a given set of nucleic acids lies within the skill of the average artisan, and suitable protocols can be found in well known literature for standard methods like, for example, “Current Protocols in Molecular Biology”, John Wiley & Sons, N.Y. (1989), 6.3.1 to 6.3.6.

Hybridisation under conditions of stringency within the different aspects of present invention is preferably understood to be:

Hybridising a labelled probe with a nucleic acid sample to be analysed at 65° C., or in the case of oligonucleotide probes, at 5° C. below the annealing or melting temperature of the duplex consisting of oligonucleotide and sample (annealing and melting temperature are in the following understood to be synonyms) over night in 50 mM Tris pH 7,5, 1M NaCl, 1% SDS, 10% Dextran Sulfate, 0.5 mg/ml denatured salmon or herring sperm DNA.

Washing for 10 minutes in 2×SSC at room temperature.

Washing for 30 minutes in 1×SSC/0.1% SDS at 65° C. (or in the case of oligonucleotides: 5° C. below the annealing temperature).

Washing for 30 minutes in 0.1×SSC/0.1% SDS at 65° C. (or in the case of oligonucleotides: 5° C. below the annealing temperature).

In one embodiment, the AS160-like protein is encoded by a nucleic acid comprising, essentially consisting of or consisting of the sequence of SEQ ID NO: 1 or of SEQ ID NO: 23. “Essentially consisting of” relates to a nucleic acid encoding a protein consisting of the sequence encoded by SEQ ID NO: 1 or 23 and short C- and/or N-terminal sequences of at most 30, 25, 20, 15, 10, 9, 8, 7, 6, 5, 4, 3, 2, or 1 amino acids homologous or heterologous to the protein. These sequences may results from the genetic manipulations, e.g. the use of particular restriction sites, or may be needed for the purification of the protein, e.g. tags such as His-tag, Strep-tag, Arg-tag, c-myc-tag or Flag-Tag.

The feature “heterologous amino acid” or “amino acid heterologous to the protein” refers to any amino acid which is different from that amino acid located adjacent to a naturally occurring As160-like protein, AS160 protein or a splice variant thereof. Therefore, the AS160-like protein encompassing at least one heterologous amino acid refers to a protein which is different from any naturally occurring AS160 protein or splice variant thereof.

A functional characteristic of novel AS 160-like protein can be any characteristic of the AS 160-like protein as specified herein. Examples of such functional characteristics encompass, but are not limited to, e.g.: its tissue distribution, its implication in insulin-stimulated signal transduction modulation such as modulation and especially stimulation of insulin-stimulated glucose uptake, a modulation and especially stimulation of the phosphorylation of AS160 and AKT, a modulation and especially stimulation of activity of PI3K (PI3-kinase) and MEKK/ERK kinases, modulation and especially stimulation of the translocation of GLUT4 to the plasma membrane, the interaction of AS160-like protein with other proteins, e.g. the interaction of AS160-like protein with GLUT4 and any other of its functional characteristics, especially as depicted in the context of this application and the experimental results given below.

According to a preferred embodiment, the test system is in a cell. A cell-based system is advantageous, because it allows for easy amplification of the test system by propagating the cells and cellular mechanisms, e.g. signal transduction components downstream of insulin or downstream or upstream of AS160-like protein, as these may be used in order to detect a signal indicative for altered glucose uptake of a cell.

Examples of cells suitable in the context of the present invention include without limitation L6 cells, 3T3 adipocytes, HEK 293, 745-A, A-431, atrial myocytes, BxPC3,

C5N, Caco-2, Capan-1, CC531, CFPAC, CHO, CHO K1, COS-1, COS-7, CV-1, EAHY, EAHY 926, F98, GH3, GP&envAM12, H-295 R, H-4-II-E, HACAT, HACAT A131, HEK, HEL, HeLa, Hep G2, High Five, Hs 766T, HT29, HUV-EC R24, HUV-EC-C, IEC 17, IEC 18, Jurkat, K 562, KARPAS-299, L 929, LIN 175, MAt-LYLU, MCF-7, MNEL, MRC-5, MT4, N64, NCTC 2544, NDCK II, Neuro 2A, NIH 3T3, NT2/D1, P19, primary neuronal cells, primary dendritic cells, primary human or mammalian myoblasts, primary adipocytes, primary keratinocytes, SF9, SK-UT-1, ST, SW 480, SWU-2 OS, U-373, U-937, rhabdomyosarcoma (RD) and Y-1. Other suitable cells are known to the one of skill in the art.

However, preferably the test system is in a cell of an insulin-dependent tissue such as adipose tissue, liver, skeletal muscle, myocardium, vascular smooth muscle and active mammary gland, preferably skeletal muscle, adipose or liver, since these are the main insulin-dependent cell types in the mammalian body. Particularly suitable cells include skeletal muscle cell, adipocyte and/or hepatocyte, as these cells might best reflect the response to a substance in the tissues relevant in type 2 diabetes.

Cells that are cultured directly from an animal or a person are known as primary cells. With the exception of some cell lines derived from tumours, most primary cell cultures have limited lifespan. After a certain number of population doublings cells undergo the process of senescence and stop dividing, while generally retaining viability.

An established or immortalised cell line has acquired the ability to proliferate indefinitely either through random mutation or deliberate modification, such as artificial expression of the telomerase gene. There are numerous well established cell lines representative of particular cell types and it is within the knowledge of the skilled person to select a suitable cell line.

Accordingly, in a preferred embodiment of the invention the cell is a cell line. A cell line is a population of cells propagated in culture that are derived from, and therefore genetically identical to, a single common ancestor cell. Preferred cell lines are L6 cells (see Examples), HEK 293 cells (primary human embryonic kidney), 3T3 cells (murine embryonic fibroblasts), CHO cells (Chinese hamster ovary), COS-7 cells (African green monkey cell line), HeLa cells (human epithelioid cervical carcinoma), JURKAT cells (human T-cell leukaemia), BHK 21 cell (hamster normal kidney, fibroblast), and MCF-7 cells (human breast cancer).

Preferred cell lines of skeletal muscle, liver or adipose tissue include without being limited thereto:

Skeletal muscle: L6 cells (see also Examples), C2C12 (mouse), preferably DSM ACC2853 (see below).

Adipose tissue: 3T3 adipocytes, brown adipocyte cell line HIB-1B), line F44-2A, those disclosed in U.S. Pat. No. 6,071,747.

Liver: BNL CL.2 (mouse, BALB/c), BNL SV A.8 (mouse), RLC-18 (rat) WRL 68.

A particularly preferred cell line encompassing a gene coding for isoforms 2 of AS160 under the control of a tetracycline-responsive promoter system (L6-GLUT4myc-tetR-AS160-like) was deposited under the Budapest Treaty on the International Recognition of the Deposit of Microorganisms for the Purposes of Patent Procedure at the “Deutsche Sammlung von Mikroorganismen und Zellkulturen GmbH” (DSMZ), Inhoffenstraβe 7 B, 38124 Braunschweig, GERMANY under the accession number DSM ACC2853 (referred to as L6-GLUT4myc-tetR-AS160-like) was deposited on Jun. 20, 2007.

In order to screen for AS160-like protein, and especially isoforms 2 of AS160-mediated effects, the results obtained with the afore-mentioned cell line may be compared to those obtained with the same cell line, but lacking a introduction of a gene coding for AS160-like protein (L6-GLUT4myc-tetR) which was also deposited under the Budapest Treaty on the International Recognition of the Deposit of Microorganisms for the Purposes of Patent Procedure at the “Deutsche Sammlung von Mikroorganismen und Zellkulturen GmbH” (DSMZ), Inhoffenstraβe 7 B, 38124 Braunschweig, GERMANY under the accession number DSM ACC2852 (referred to as L6-GLUT4myc-tetR) on Jun. 20, 2007.

Both cell lines (L6-GLUT4myc-tetR-AS160-like and L6-GLUT4myc-tetR) have been prepared as detailed in Example 2. Briefly summarized, the tetracycline-repressor (TR) was isolated from pCDNA3.1 (+)/TR (Invitrogen), cloned into the Nhel and Notl sites of pIRESpuro2 as shown in FIG. 3 to obtain pIRESpuro2/TR which was transfected into L6 cells (rat skeletal muscle cells) stably expressing GLUT4myc (L6-GLUT4myc, described in Wang et al. 1998). For L6-GLUT4myc-tetR-AS160-like cells, pCDNA5 vector (Invitrogen) containing the AS160, isoforms 2 gene was additionally introduced into the cells.

Analogously, a cell line expressing isoforms 3 of AS160-like protein can be prepared using similar procedures according to standard protocols known to the person skilled in the art.

For cultivation, cells may be grown and maintained at an appropriate temperature and gas mixture (typically, 37° C., 5% CO₂) in a cell incubator. Culture conditions vary widely for each cell type, and variation of conditions for a particular cell type can result in different phenotypes being expressed. Aside from temperature and gas mixture, the most commonly varied factor in culture systems is the growth medium. Recipes for growth media can vary in pH, glucose concentration, growth factors, and the presence of other nutrient components. Antibiotics can also be added to the growth media. Amongst the common manipulations carried out on culture cells are media changes and passaging cells. However, selection of suitable conditions is known to the skilled person.

The cell or cell line may be genetically engineered to include the test system of the invention. The test system may be located in a transient or stable transfected cell or cell line. The procedure for introducing a transgene into a recipient cell is called transfection. Transfection with DNA yields stable as well as unstable (transient) cells or cell lines. Transient cell lines reflect the survival of the transfected DNA in extrachromosomal form; stable cell lines result from the integration into the genome.

The transgenes can be introduced into the cells by a variety of means known to those knowledgeable in the art, and adapted to each cell type. Recombinant DNA cloning techniques well known in the art for introducing and expressing a nucleic acid molecule can be used to introduce and express the transgenes. Cells can be transfected using any appropriate means, including viral vectors, chemical transfectants, electroporation, calcium phosphate co-precipitation and direct diffusion of DNA. A suitable method for introducing a tests system into a recipient cell is detailed in Example 2 and may be adapted to the respective recipient cell.

As used herein, vectors are agents that transport the transgene into the cell and may include appropriate transcriptional and translational control signals such as a promoter. Vectors can be plasmid, viral or others known in the art. The promoter can be inducible or constitutive, general or cell specific, nuclear or cytoplasmic specific promoter. Selection of promoters, vectors and other elements is a matter of routine design within the level of ordinary skill in the art. Many such elements are described in the literature and are available through commercial suppliers. Usually, the method of transfer includes the transfer of a selectable marker to the cells. Suitable promoters and vectors are disclosed in the Examples and the present description.

In general, a cell line is transfected by any of the means mentioned above, wherein the transgene is operatively linked to a selectable marker. Following transfection cells are grown e.g. for some days in enriched media and then switched to selective media. Transfected cells exhibit resistance to the selection and are able to grow, whereas non-transfected cells die in general. Examples for selective markers include puromycin, zeocin, neomycin (neo) and hygromycin B, which confer resistance to puromycin, zeocin, aminoglycoside G-418 and hygromycin, respectively, and/or any of the selective markers used in the Examples. However, other selection methods known to the skilled person may be also suitable.

In the step b) of the method of the present invention a test substance is identified as a substance altering glucose uptake of a cell by detecting a signal indicative for altered glucose uptake of a cell. The signal may be any suitable signal which is indicative for altered glucose uptake of a cell; however, the signal may by any component or part of the insulin-stimulated signal transduction relating to AS160-like protein. Particularly, it may be the degree of phosphorylation of AS160 or AKT, activity of PI3K (PI3-kinase) or MEKK/ERK kinases, translocation of GLUT4 to the plasma membrane or increase in glucose uptake of a cell.

Suitable methods for measuring the aforementioned components of the AS160-like protein signal transduction pathway are known in the art and are also detailed in the Examples.

Preferably, the detectable signal is the amount of AS160-like protein (isoforms 2 and/or 3) expressed in a cell, phosphorylated AKT, phosphorylated AS160-like protein, GLUT4 translocation to the plasma membrane, GLUT4 distribution in a cell or glucose uptake by a cell. As could be shown in the examples, all these signals correlate with the glucose uptake of a cell, preferably a cell of an insulin-sensitive tissue.

The detectable signal may be the amount of AS160-like protein in a cell, as the amount of this protein is indicative for glucose uptake. If the amount of this protein is increased, the glucose uptake of a cell, particularly an insulin-sensitive cell, is increased, too. Methods of determining the amount of a particular protein are known to the skilled person and include e.g. Western blotting and detection with specific antibodies, which may be carried out as detailed in the Examples. A specific antibody is also provided in the Examples. Alternatively, an anti-AS160-like monoclonal or polyclonal antibody may be produced in accordance with the knowledge of the skilled person and detected by enzyme-linked immunosorbent assay (ELISA), radioimmunoassay (RIA), and fluorescence activated cell sorting (FACS).

A variety of other techniques known in the art can be used to quantify the amount of a given protein. These include, but are not limited to immunological techniques such as an ELISA or RIA, or quantitative analytical techniques such as spectroscopy or flame chromatography. Alternatively, the amount of AS160-like-mRNAs could be determined by a hybridization method or a nucleic acid amplification method instead of the amount of protein. Such methods are known to the artisan and include the dot blot hybridization method, the Northern hybridization method or the RT-PCR method.

An alternative detectable signal may be the amount of phosphorylated AKT and/or phosphorylated AS160-like protein, as the degree of phosphorylation of these proteins is indicative for glucose uptake. If the amount or degree of phosphorylation is increased, the glucose uptake of a cell, particularly an insulin-sensitive cell, is increased, too. Methods of determining the amount or degree of phosphorylation are known to the skilled person and include the use of antibodies specific for these phosphorylated proteins as detailed in the Examples.

A further detectable signal may be GLUT4 translocation to the plasma membrane or GLUT4 distribution in a cell, as the degree of translocation of GLUT4 is indicative for glucose uptake. If the amount or GLUT4 in the plasma membrane is increased, the glucose uptake of a cell, particularly an insulin-sensitive cell, is increased, too. Methods of determining GLUT4 translocation to the plasma membrane or GLUT4 distribution in a cell are known to the skilled person and include the use myc-tagged GLUT4 in an In-cell-Western technique or in a ACUMEN technique. These methods may be carried out as detailed in the Examples.

Alternatively, glucose uptake of a cell could be determined, e.g. by using labeled glucose or a labeled glucose derivative. Suitable labels include e.g. detectable tags, radio-active isotopes such as ³H or ¹⁴C or fluorescence markers. Such labeled glucose or a labeled glucose derivatives include without limitation 2-fluoro-2-deoxy-D-glucose, 2-deoxy[¹⁴C] glucose and [¹⁴C]methylglucose. Preferably, radio-labeled 2-deoxyglucose is used. This method may be carried out as detailed in the Examples.

As detailed above, the method of the invention may be used in order to test as substance under high glucose condition, which better reflects the situation in a patient suffering from diabetes. High glucose conditions are those with elevated glucose concentration. The normal/safe level for glucose in the blood of a human is between 3.5 and 7.8 mM. Accordingly, a high glucose condition is a condition with glucose concentration above the normal level. Particularly, the glucose concentration used for the method of the invention may be at least 10 mM, preferably at least 15 mM, more preferably at least 25 mM glucose.

The substance tested with the method of the invention may be any test substance or test compound of any chemical nature. It may be already known as a drug or medicament for a disease other than type 2 diabetes. Alternatively, it may be a known chemical compound not yet known to have a therapeutic effect. In another embodiment the chemical compound may be a novel or so far unknown chemical compound.

In another embodiment of the screening methods of the invention, the test substance is provided in the form of a chemical compound library. Chemical compound libraries include are plurality of chemical compounds and have been assembled from any of multiple sources, including chemically synthesized molecules and natural products, or have been generated by combinatorial chemistry techniques. They are especially suitable for high throughput screening. They may be comprised of chemical compounds of a particular structure or compounds of a particular creature such as a plant. In the context with the present invention the chemical compound library is preferably a library comprising proteins and polypeptides or small molecules.

Advantageously, the method of the present invention is carried out in a robotics system e.g. including robotic plating and a robotic liquid transfer system, e.g. using microfluidics, i.e. channelled structured.

In another embodiment of the present invention, the method is carried out in form of a high-through put screening system. In such a system advantageously the screening method is automated and miniaturized; in particular it uses miniaturized wells and microfluidics controlled by a roboter. High-throughput screening (HTS), is a method for scientific experimentation especially used in drug discovery and relevant to the fields of biology and chemistry.

HTS allows a researcher to effectively conduct millions of biochemical, genetic or pharmacological tests in a short period of time, often through a combination of modern robotics, data processing and control software, liquid handling devices, and sensitive detectors. Through this process one can rapidly identify active compounds which modulate a particular biomolecular pathway; particularly a substance altering the glucose uptake of a cell.

In essence, HTS uses an approach to collect a large amount of experimental data on the effect of a multitude of substances on a particular target in a relatively short time. A screen, in this context, is the larger experiment, with a single goal (usually testing a scientific hypothesis), to which all this data may subsequently be applied.

For HIS, cells comprising AS160-like protein or a nucleic acid coding for the same may be seed in a tissue plate, such as a multi well plate, e.g. a 96-well plate. Then the cell in the plate is contacted with the test substance for a time sufficient to stimulate and generate a suitable detectable signal as defined above. The test substance may be different from well to well across the plate. After incubation time has passed to allow generation of the signal, measurements are taken across all the plate's wells, either manually or by a machine.

Manual measurements may be necessary when the researcher is using microscopy to (for example) seek changes the wells' test compounds, looking for effects that a computer could not easily determine by itself. Otherwise, a specialized automated analysis machine can run a number of experiments on the wells (such as analyzing light of a particular frequency). In this case, the machine outputs the result of each experiment e.g. as a grid of numeric values, with each number mapping to the value obtained from a single well.

Depending upon the results of this first assay, the researcher can perform follow up assays within the same screen by using substances similar to those identified as active (i.e. altering glucose uptake of a cell) into new assay plates, and then re-running the experiment to collect further data, optimize the structure of the chemical compound to improve the effect of the compound on the cell.

Automation is an important element in HTS's usefulness. A specialized robot is often responsible for much of the process over the lifetime of a single assay plate, from creation through final analysis. An HTS robot can usually prepare and analyze many plates simultaneously, further speeding the data-collection process.

A further subject of the invention relates to a test system for the identification of a substance for improving glucose uptake into a cell, the test system comprising

a gene coding for the AKT substrate 160 kDa-like protein (AS160-like protein) or functional variant thereof; and

an inducible promoter providing controllable expression of the gene,

wherein the activation of AS160-like protein effects a detectable signal.

It is noted that all features of this test system may be further defined as detailed in connection with the method of the invention.

The test system is particularly useful as it allows for identification of a substance improving (i.e. increasing) glucose uptake of a cell or as a model for studying type 2 diabetes. The combination of AS160-like protein and an inducible promoter providing controllable expression of the gene allows for determining effects in identical cells with and without AS160-like protein. Accordingly, differences in signaling obtained in cells expressing AS160-like protein in comparison to those not expressing AS160-like protein may be assigned to AS160-like protein. As AS160-like protein may be used to identify new potential drugs for type 2 diabetes (as detailed above) or as a key protein in the main insulin-sensitive tissues, these test system may be used to obtain news insights in the pathophysiology and therapy of type 2 diabetes.

Preferably, the test system of the invention is located in a cell, particularly a genetically engineered cell. The cell any be any of the cells disclosed in the context of the method of the invention. Particularly the gene and/or the promoter may be introduced into the genetically engineered cell.

The test system of the invention comprises an inducible promoter providing controllable expression of the gene. Controllable expression of the gene means that the expression can be induced or repressed upon a chemical or physical stimulus to the test system which can be applied as intended by the investigator or experimenter.

Promoters represent critical elements that can work in concert with other regulatory regions (enhancers, silencers, boundary elements/insulators) to direct the level of transcription of a given gene. An inducible promoter is activated in response to either the presence of a particular compound, i.e. the inducer (chemical stimulus) or to a defined physical condition, e.g. elevated temperature (physical stimulus). Inducible promoters are a very powerful tool in genetic engineering because the expression of genes operably linked to them can be turned on or off as desired.

There are a series of chemically-regulated promoters, including promoters whose transcriptional activity is regulated by the presence or absence of alcohol, tetracycline, steroids, metal and other compounds. Physically-regulated promoters include promoters whose transcriptional activity is regulated by the presence or absence of light and low or high temperatures.

Preferably, chemically-regulated promoters should be derived from organisms distant in evolution to the cell where its action is required. Thus, promoters to be used in mammalian cells are mostly derived from organisms such as yeast, E. coli or

Drosophila. Particular examples are alcohol-regulated promoter system (alcohol dehydrogenase I (alcA) gene promoter and the transactivator protein AIcR); tetracycline-regulated promoter system (tetracycline repressor protein (TetR), tetracycline operator sequence (tetO), tetracycline transactivator fusion protein (tTA), which is the fusion of TetR and a herpes simplex virus protein 16 (VP16) activation sequence, the promoter system disclosed in Example 2); steroid-regulated promoter systems (steroid-responsive promoter, e.g. promoters based on the rat glucocorticoid receptor (GR) or promoters based on the human estrogen receptor (ER)); or metal-regulated promoters derived from metallothionein genes from yeast, mouse and human.

However, the inducible promoter is preferably a tetracycline-inducible promoter, more preferably the promoter system as described in Example 2.

A further aspect of the invention relates to the use of a tests system comprising AS160-like protein for the identification of a substance altering, particularly improving, glucose uptake into a cell as already detailed above in the context of the method are test system of the invention. The test system used may be further specified as described in above with respect to the method or test system of the invention.

Also in accordance with the above disclosure AS160-like protein may be used in a model for type 2 diabetes, wherein the above details with respect to the method or test system of the invention are to be applied accordingly.

A further embodiment of the invention concerns a polypeptide consisting or essentially consisting of the amino acid sequence according to SEQ ID NO: 3 or 23 or being encoded by a sequence according to SEQ ID NO: 1 or 22.

In another embodiment, the invention concerns a polynucleotide consisting of or essentially consisting of polynucleotide sequence according to SEQ ID NO: 1 or 22 or encoding a polypeptide according to SEQ ID NO: 3 or 23.

Yet another embodiment of the invention concerns an antibody or functional fragment thereof, specifically binding to a polypeptide consisting or essentially consisting of the amino acid sequence according to SEQ ID NO:3 or 23 or being encoded by a sequence according to SEQ ID NO:1 or 22.

The preparation of suitable antibodies or functional fragments thereof is well known in the art, e.g. by immunizing a mammal, for example a rabbit, with AS 160-like protein or a fragment thereof, where appropriate in the presence of, for example, Freund's adjuvant and/or aluminium hydroxide gels (see, for example, Diamond, B.A. et al. (1981) The New England Journal of Medicine: 1344-1349). The polyclonal antibodies which are formed in the animal as a result of an immunological reaction can subsequently be isolated from the blood using well known methods and, for example, purified by means of column chromatography. Suitable procedures to produce monoclonal antibodies are well known in the art as well (see e.g. Winter, G. & Milstein, C. (1991) Nature, 349, 293-299 and literature for standard methods listed below). In the context of present invention, the term antibody or antibody fragment comprises also recombinant antibodies or antigen-binding parts thereof, e.g. chimaeric, humanized, multifunctional, bispecific, oligospecific or single-stranded antibodies or antibody F(ab) or F(ab)₂ fragments (see, e.g. EP-B1-0 368 684, WO 88/01649, WO 93/06213, WO 98/24884, U.S. Pat. No. 4,816,567 or U.S. Pat. No. 4,816,397).

A specific anti AS 160-like antibody according to the invention should interact more strongly with AS160-like protein than with the isoform 1 of AS160 under standard laboratory conditions (e.g. in a Western Blot or the like). According to one embodiment, the specific anti AS 160-like antibody interacts more strongly with novel isoform 2 of AS 160, as identified herein, than with one or both of the isoforms 1 or 3 of AS 160 protein under standard laboratory conditions (i.e. a specific AS 160, isoform 2 antibody). According to another embodiment, the specific anti AS 160-like antibody according to the invention interacts more strongly with isoform 3 of AS160 than with one or both of the isoforms 1 or 2 of AS160 protein under standard laboratory conditions (i.e. a specific AS 160, isoform 3 antibody).

According to another embodiment, the invention concerns a cell heterologously expressing a polypeptide consisting or essentially consisting of the amino acid sequence according to SEQ ID NO: 3 or 23 or being encoded by a sequence according to SEQ ID NO: 1 or 22 or a cell stably or transiently transfected with a polynucleotide consisting of or essentially consisting of polynucleotide sequence according to SEQ ID NO: 1 or 22 or encoding a polypeptide according to SEQ ID NO: 3 or 23.

The cell can be any procaryotic or eucaryotic cell capable of being stably or transiently transfected with a nucleic acid vector and of expressing a heterologous gene. These comprise principally primary cells as well as cells from a cell culture, preferably a eucaryotic cell culture comprising cells derived either from multicellular organisms and tissue (such as HeLa, CHO, COS, SF9 or 3T3 cells) or from single cell organisms such as yeast (e.g. s. pombe or s. cerevisiae), or a procaryotic cell culture, preferably Pichia or E. coli. Cells and samples derived from tissue can be gained by well-known techniques, such as taking of blood, tissue punction or surgical techniques.

Another aspect of the invention concerns a siRNA (small inhibitory RNA) capable of negatively interfering with expression and/or activity of any of AS160 like isoforms 2 and/or 3, e.g. specific for or in part complementary to at least a part of the DNA sequence SEQ ID NO:1 or SEQ ID NO.22:

The term “siRNA” refers to small inhibitory RNAs that induce the RNA interference (RNAi) pathway (for the RNAi interference pathway, see e.g. Elbashir et al., Genes and Development (2001) 15: 188-200, Tuschl. et al., (1999), Genes and Development, 13: p. 3191-3197 or Zamore et al, Cell (2000) vol.101, p.25-33). In the context of present invention the term “siRNA” comprises duplexes of two separate strands, as well as single strands that can form hairpin strucures comprising a duplex region, so-called shRNAs—short hairpin RNAs. siRNA molecules can vary in length (in general 15 to 35, 18 to 30 or 20 to 25 nucleotides in length); however, the choice of the appropriate length is well known in the art. Moreover, siRNAs can vary in their degree of complementarity to their target mRNA in the antisense strand. The choice of the appropriate degree of complementarity is also well known in the art. siRNAs may have unpaired overhanging bases on the 5′ and/or the 3′ end of the sense strand and/or the antisense strand.

Design and preparation of siRNAs for a given cDNA sequence are well known in the art see, for example Elbashir et al. (2001) Nature 411: 494-498 (see especially p.497, right column, for preparation of siRNA and siRNA transfection into cells), and Tuschl et al. (1999) Genes and Development 13: 3191-3197).

Methods for application of siRNA include chemically synthesized or in vitro transcribed siRNA, e.g. duplex or shRNA, which are than to be transfected or injected into cells or transgenic animals. SiRNA can also be expressed from expression vectors or PCR products in cells or transgenic animals, wherein the term expression vector refers to any kind of vector system useful for driving expression of siRNAs (either duplex or shRNA) and comprises shuttle vectors as well as viral, such as retro-and lentiviral vectors, as well known in the art.

According to another aspect, the invention refers to the use of the AS 160-like protein (isoforms 2 and or 3) or the nucleic acid sequence thereof, for generating a siRNA, either duplex or shRNA, able to negatively interfere with expression and/or activity of AS 160-like protein (isoforms 2 and/or 3).

The following figures and examples shall illustrate the present invention, but should not be understood as limiting the scope of the invention.

### FIGURES

FIG. 1 shows a comparison of the three different isoforms of AS160. Novel AS160-like protein (isoform 2 as well as novel isoform 3) has been identified on the basis of quantitative RT PCR (Taqman) using primers SEQ ID NO 7, 8, 9 for isoform 2. Full-length AS160 (amplified with primers SEQ ID NO 19, 20 and 21) is depicted in (A), the AS160-like protein, isoform 2, which lacks the exons 11 and 12 in (B). Isoform 3 is shown in (C) (amplified with primers SEQ ID NO 16, 17, 18). Phosphorylation sites Ser-588 and Thr-642 (underlined) and mismatches T202 and T1275 (italic) are shown.

FIGS. 2(A and B) shows expression of AS160 isoforms (Taqman). Using specific primers (Primers SEQ ID NO 4, 5, 6 and/or 19, 20, 21) for full-length AS160 , primers SEQ ID NO 7,8, 9 for AS160-like and primers 16, 17, 18 for isoform 3), the expression of the three different isoforms was examined. mRNA levels of AS160 isoforms are normalized against expression of endogenous RPL37a mRNA (amplified with primers SEQ ID NO 10, 11, 12). Data are representative for five different human donors.

FIG. 3 shows a schematic presentation of cloning strategy for cloning of isoforms 2 of AS160. The tet-repressor derived from pCDNA3.1 (+)/TR was cloned into Nhel and NotI sites of pIRESpuro2 generating pIRESpuro2/TR.

FIG. 4 shows that expression of isoforms 2 of AS160 in L6-GLUT4myc cells is tet-inducible. RIPA (radio immuno precipitation assay) extracts were analyzed with SDS-PAGE and western blot analysis. Cells were incubated in the absence of doxycycline (−isoform2) or in the presence of doxycycline (+isoform2) for 48 hours. Expression of AS160-like was detected with a specific AS160 antibody (Upstate, Cat No.: 07-741).

FIG. 5 shows insulin stimulated AKT activation in L6-GLUT4myc cells. with or without expression of isoforms 2 of AS160. Phosphorylation of AKT at Ser 473 was confirmed with an anti-pAKT (Ser 473) antibody (Biosource, Cat No.: 44-621G).

FIG. 6 shows phosphorylation of AKT and isoform2 of AS160 (In-cell western). An In-cell western analysis was performed in a 96 well plate. Cells were pre-treated with doxycyclin for 48 hours to induce the expression of isoform2 of AS160 (+isoform2 of AS160). Insulin was incubated for 20 minutes. The phospho-AKT antibody detects the phosphorylation of AKT at Ser 473 (Biosource, Cat No.: 44-621G). The phospho-AS160 antibody (Biosource Cat No.: 44-1071G) is specific for the phosphorylation site at Thr 642. Standard deviations represent 8 reading points. *P values<0.001 vs.−AS160-like. RU represents relative units.

FIG. 7 shows the effect of isoform2 of AS160 on insulin stimulated glucose uptake. Uptake of 2-deoxyglucose is measured in response to insulin. Insulin was incubated for 20 minutes. Standard deviations are representative for 8 reading points. *P<0.001vs.−isoform2 of AS160-like values.

FIG. 8 shows dose-dependency of glucose uptake. Doxycyclin-dependency was investigated with uptake of 2-deoxyglucose of L6-GLUT4myc cells containing isoform2 of AS160. Doxycyclin was incubated for 48 hours. Insulin was incubated for 20 minutes. Standard deviations are representative for 8 reading points.

FIG. 9 shows the effect of isoform2 of AS160 on IGF-1 and AICAR stimulated glucose uptake. Uptake of 2-deoxyglucose is measured in response to IGF-1 (A) and AICAR (B). IGF-1 was incubated for 20 minutes. AICAR was incubated for 2 hours. Standard deviations are representative for 4 to 8 reading points. *P<0.05 vs.−isoform2-values.

FIG. 10 shows the effect of isoform2 of AS160 on metformin-stimulated glucose uptake. Uptake of 2-deoxyglucose is measured in response to insulin (left) and metformin (right). Metformin was incubated during starvation period (3 hours), insulin was incubated for 20 minutes. Standard deviations are representative for 4 reading points. *P<0.05 vs.−AS160-like values, **P<0.001 vs.−AS160-like values.

FIG. 11 shows the effect of AKT inhibitors. 10 μg RIPA extract were separated by SDS-PAGE and analyzed by western blotting with a pAKT (Ser 473) specific antibody (Biosource, Cat No.: 44-621G) (A). Cells were pre-incubated with either wortmannin (200 nM; Upstate, Cat No.: 12-338) or the AKT inhibitor Calb. (50 μM, Calbiochem, Cat No.: 124005) for 1 hour and subsequently stimulated with insulin (10 nM) for 20 minutes. Uptake of 2-deoxyglucose (B) was measured in response to insulin and AICAR alone and in combination of wortmannin plus insulin or AICAR (Biomol). Insulin was incubated 20 minutes, AICAR was incubated for 2 hours. *P<0.001 vs.−10 nmol insulin.

FIG. 12 shows the time-dependency of glucose uptake. The uptake of 2-deoxyglucose was measured after 5, 15, 30 minutes. Cells were pre-treated with doxycyclin in order to induce expression of AS160-like protein or left untreated. Standard deviations were obtained from 8 independent values.

FIG. 13 shows the effect of MEKK/ERK inhibitor on glucose uptake. Expression of isoform2 of AS160 was induced with doxycyclin for 48 hours (+AS160-like). Cells were starved for 4 hours. The MEKK/ERK inhibitor U0126 (10 μM, 20 μM, Upstate Cat No.: 19-147) was incubated in starve medium. Subsequently, cells were stimulated for 20 minutes with the indicated insulin concentrations. *P<0.05 vs.—AS160-like.

FIG. 14 shows the induction of insulin-resistance (glucose uptake). To induce insulin-resistance cells were incubated in the presence of high glucose and insulin overnight (right), or incubated under normal conditions (left). Induction of isoform2-expression was performed by incubation of doxycyclin for 48 hours. Insulin was incubated for 20 minutes. Standard deviations represent 8 reading points. *P<0.05 vs.−isoform2 of AS160.

FIG. 15 shows induction of insulin-resistance (In-Cell western). To induce insulin-resistance cells were incubated in the presence of high glucose plus insulin overnight or incubated under normal conditions. Induction of isoform2-expression was performed by incubation of doxycyclin for 48 hours. Activation of AKT was determined with an antibody recognizing Ser 473 (Biosource Cat No.: 44-621G). The phospho-AS160 antibody is directed against the Thr 642 site. *P<0.05 vs.100 nM insulin under normal conditions.

FIG. 16 shows the effect of metformin on glucose uptake. Expression of isoform2 of AS160 in all wells was induced with doxycyclin for 48 hours. Insulin-resistance was induced overnight. Metformin (800 μM) was incubated overnight. Cells were starved for 4 hours and subsequently stimulated with different insulin concentrations under indicated conditions. *P<0.05 vs. appropriate values without compound.

FIG. 17 shows the effect of metformin on phosphorylation of AKT and AS160, isoform 1 (In-cell Western). Expression of isoform2 of AS160 in all wells was induced with doxycyclin for 48 hours. Insulin-resistance was induced overnight. Metformin (800 μM) was incubated overnight. Cells were starved for 4 hours and subsequently stimulated with different insulins with the indicated conditions. RU represents relative is units.

FIG. 18 shows an analysis of GLUT4 translocation (ACUMEN). Cells were incubated with doxycyclin to induce expression of AS160-like protein or left untreated. Insulin was incubated for 20 minutes. Standard deviations were obtained from 8 single values. *P<0.05 vs. L6-GLUT4-myc+100 nM insulin. #P<0.05 vs.−isoform2 of AS160+100 nM insulin.

FIG. 19 shows a graphic presentation of GLUT4 distribution (ACUMEN). Histograms were obtained on the basis of laser-scanning fluorescence cytometry after different conditions. The curves represent total cell counts (DNA marker). Cells left of the arrow shown in lighter grey are negative for myc-GLUT4 staining. Cells right of the arrow shown in lighter grey represent the cell population that display translocated myc-tagged GLUT4 at the plasma membrane.

FIG. 20 shows the location of the forward and reverse primers for PCR-cloning of isoform2 of AS160 from human testis cDNA on the DNA sequences upstream and downstream of the isoform2-coding Sequence (SEQ ID NO:13). Primers are typed bold and underlined by dotted lines; the AS160-like coding sequence is underlined by a solid line.

FIG. 21 shows an immunofluorescence based analysis of the intracellular localization of myc-GLUT4 and isoform2 of AS160 under basal and insulin stimulated conditions. Rat myoblast cells were incubated with insulin (100 nM) for 20 minutes prior to treatment for immunofluorescence-microscopy.

### EXAMPLES

### Example 1

### Expression of AS160 Isoforms in Different Tissues

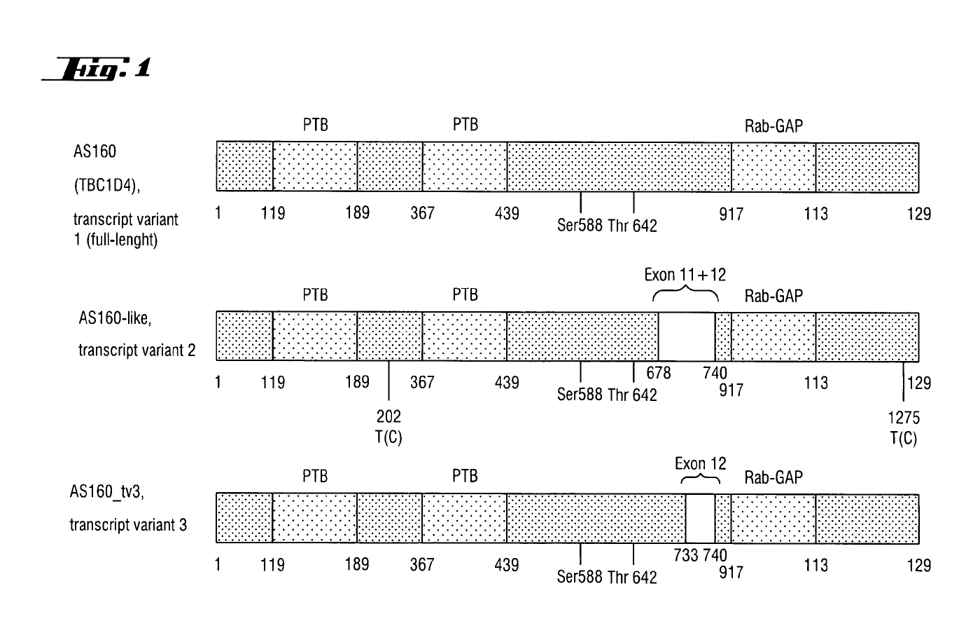

Bioinformatical analysis of ESTs indicated the presence of three isoforms of AS160 (FIG. 1A, B, C). FIG. 1 presents a schematic overview of AS160 and the new isoforms identified. The expression of these different AS160-isoforms was investigated with quantitative RT-PCR in different tissues. Commercially available RNAs of five different human donors were reverse transcribed and examined using Taqman PCR with specific primer pairs (for full-length AS160 SEQ ID NO: 4, 5 and 6, (or alternatively 19, 20 and 21) for AS160-like SEQ ID NO: 7, 8 and 9, for isoform 3 SEQ NO: 16, 17 and 18).

For this, aliquots of total cellular RNA were subjected to first-strand DNA synthesis. Reverse-transcribed cDNA was used as a template for amplification. A common probe was used to determine the overall AS160 expression in insulin-sensitive tissue (adipose, muscle, liver, heart, brain). Endogenous mRNA expression of the ribosomal gene RPL37a (Homo sapiens ribosomal protein L37a, mRNA, cDNA clone MGC:26772) was used to normalize mRNA levels (SEQ ID NO: 10, 11 and 12). Based on specific primer pairs the expression of distinct isoforms could be distinguished. Relative mRNA expression methods were calculated with the deltadelta CT method (Yuan et al., 2006).

The following primers and probes were used:

| | | SEQ |
|---|---|---|
| | | ID |
| Specificity | Sequence | NO: |
| Isoform 1 | For 5′-ATTCAGGTAGACTGTCCCCACAGTAT | 19 |
| (full-length | | |
| AS160) | | |
| | Rev 5′-CCTTCTCCATCACTTGATTCTGAAG | 20 |
| | Probe: FAM-MGBNFQ | 21 |
| | 5′-ATGAAATCAGACAAGACACTG | |
| Isoform 2 | For 5′-GCGTTCCCCTCTGCTGAG | 7 |
| (AS160-like) | | |
| | Rev 5′-ACTCATTGCTGCAGGTAGATGAG | 8 |
| | Probe: FAM-TAMRA | 9 |
| | 5′-TTCTCCATCACTGCACTGTTCACTGGAGCT | |
| RPL37a | For 5′-ACAGCGGAAGTGGTATTGTACGT | 10 |
| | Rev 5′-GGCACTGTGGTTCCTGCAT | 11 |
| | Probe: VIC-TAMRA | 12 |
| | 5′-CAGGCACCGCCAGCCACTGTCT | |
| PCR cloning | For 5′-GGAGGAGGATGCCCATTTAAC-3 | 14 |
| of Isoform2 | (SEQ ID NO: 14) | |
| cDNA | | |
| | Rev 5′-TCTAAGGAGCACTTTCTGCTGAG-3′ | 15 |
| Isoform 3 | For 5′-AGCTTTTACCAGAATTCAGGTAGACTG | 16 |
| | T | |
| | Rev 5′-TGCTGCAGGTAGATGAGGTCCT | 17 |
| | Probe: FAM-MGBNFQ | 18 |
| | 5′-CTTCTCCATCACTGATTTCATT | |
| Isoform1 | For 5′-CATACTCTTCTTAAAGAAGGAGTTCCCA | 4 |
| (full-length | | |
| AS160) | | |
| | Rev 5′-CTGTGTCTGAGTCGGTACTGTAAAGC | 5 |
| | Probe: FAM-TAMRA | 6 |
| | 5′-CAGAAACTGCCAAATTTCTCCTCGTCGACT | |

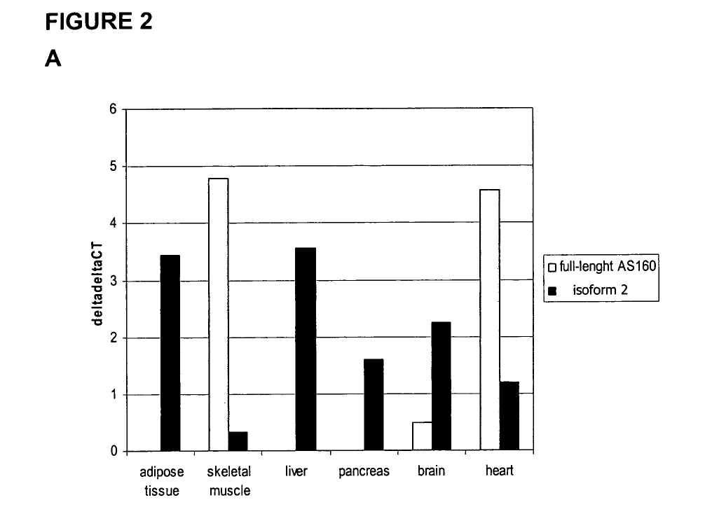

The results of this RT-PCR are shown in FIG. 2. Isoform 1 (NM_{—}014832_v1; EMBL) represents full-length AS160. Full-length AS160 as well as isoform 3 are mainly expressed in heart and skeletal muscle (FIGS. 2A and B). Isoform 2, which represents the AS160-like, is mostly expressed in adrenal and thyroid glands, but also in lung kidney and brain (Figure B). Expression is also detected in adipose tissue and in the liver (FIG. 2A). In comparison to full-length AS160 and isoform 3, AS160-like is only slightly expressed in skeletal muscle. The data might indicate a specific function of distinct isoforms in different tissues. In the following experiments the inventors focused on the examination of isoform 2 (AS160-like), since it is novel and shows highest overall expression in most tissues.

### Example 2

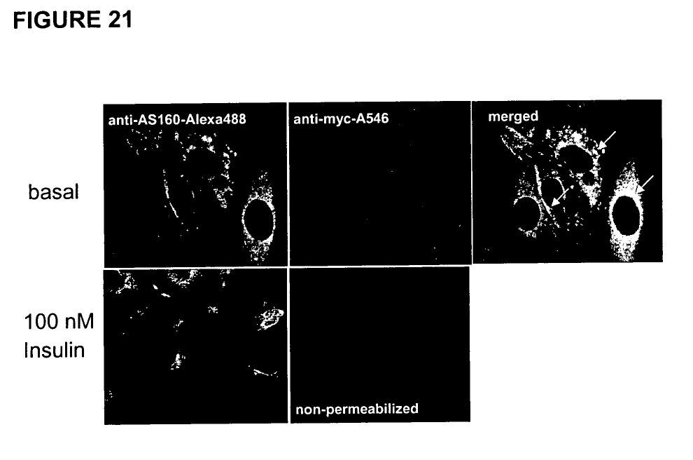

Cloning of novel AS160-like protein expression construct and establishment of a tetracycline-inducible AS160-like protein expression system AS160-like insert (SEQ ID NO: 1) was amplified from human testis cDNA (Clontech, Cat# 7117-1; Lot#2100009) using primers according to FIG. 21 (SEQ IDs 14 and 15); the obtained PCR fragment (see FIG. 21, the insert is underlined) was used as a template for generating a fragment corresponding to the coding sequence having gateway sequences for cloning. The insert was cloned into pDONR221 (Invitrogen) and subsequently into the expression vector pCDNA5-TO (Invitrogen) by means of standard methods. Sequencing analysis revealed that in comparison to full-length AS160 (NM_{—}014832; EMBL) exons 11 and 12 are missing in AS160-like (FIG. 1). Two mismatches were identified at positions nt606 (silent) and nt3827 (Ala Val). In addition, this clone contains a 3 by deletion (nt 2594-2596), that was also found in human placenta cDNA but not in human brain cDNA. For the expression clone of the isoform lacking exons 11 and 12 the 3bp deletion was repaired to resemble more closely the full length sequence of NM_{—}014832 (EMBL).

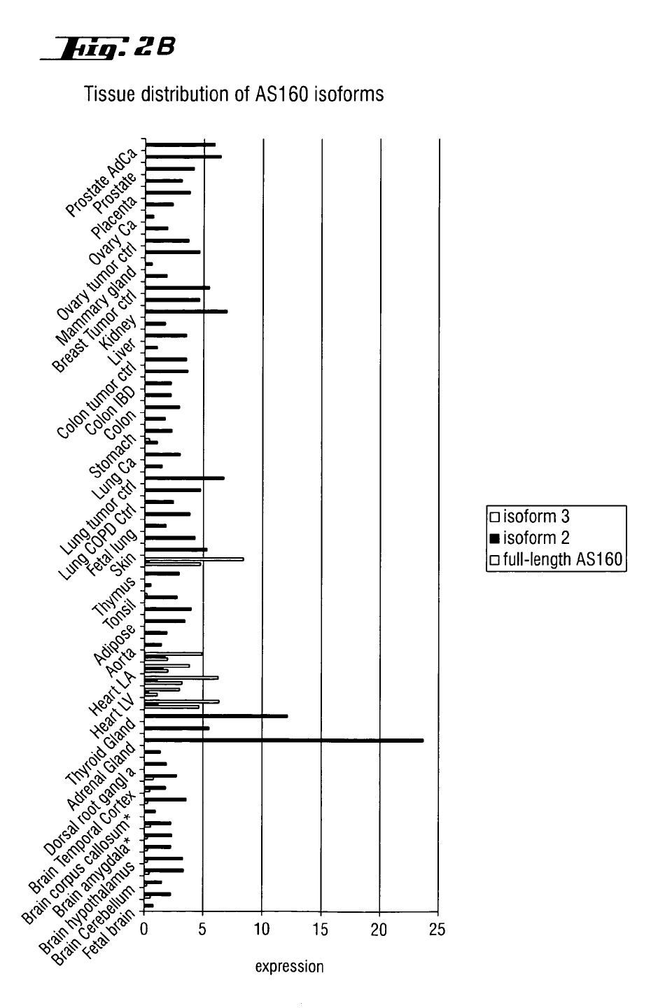

L6 myoblasts (rat skeletal muscle cells) stably expressing glucose transporter 4 (GLUT4) with an exofacially directed myc-tag (GLUT4myc) (L6-GLUT4myc, described in Wang et al. 1998) were subsequently used for tetracycline (tet) -inducible expression of AS160-like protein. For this purpose the T-REx system from Invitrogen was used. The regulatory plasmid in this system controls the constitutive expression of the tet-repressor (tet-R) under the control of a CMV (cytomegalovirus) promoter. In the absence of tetracyline (or doxycycline) the repressor binds to specific tetracycline-operator sequences (TetO2) and thereby represses expression. Addition of tetracycline (or doxycycline) induces expression of the protein of interest. To allow a stable integration of the tet-system in L6-GLUT4myc cells the tet-repressor was isolated from pCDNA3.1 (+)/TR (Invitrogen) and cloned into the Nhel and Notl sites of pIRESpuro2 as shown in FIG. 3. The sequence of pIRES-puro2/TetR is given in the following as SEQ ID NO: 2.

Sequence: pIRES-puro2/TetR (SEQ ID NO: 2)

GACGGATCGGGAGATCTCCCGATCCCCTATGGTCGACTCTCAGTACAATC
TGCTCTGATGCCGCATAGTTAAGCCAGTATCTGCTCCCTGCTTGTGTGTT
GGAGGTCGCTGAGTAGTGCGCGAGCAAAATTTAAGCTACAACAAGGCAAG
GCTTGACCGACAATTGCATGAAGAATCTGCTTAGGGTTAGGCGTTTTGCG
CTGCTTCGCGATGTACGGGCCAGATATACGCGTTGACATTGATTATTGAC
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGACTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTCTCTGGCTAACTAGAGAACCCACTGCTTACTG
GCTTATCGAAATTAATACGACTCACTATAGGGAGACCCAAGCTTGGTACC
GAGCTCGGATCGATATCTGCGGCCTAGCTAGCGTTTAAACTTAAGCTTAC
CATGTCTAGATTAGATAAAAGTAAAGTGATTAACAGCGCATTAGAGCTGC
TTAATGAGGTCGGAATCGAAGGTTTAACAACCCGTAAACTCGCCCAGAAG
CTAGGTGTAGAGCAGCCTACATTGTATTGGCATGTAAAAAATAAGCGGGC
TTTGCTCGACGCCTTAGCCATTGAGATGTTAGATAGGCACCATACTCACT
TTTGCCCTTTAGAAGGGGAAAGCTGGCAAGATTTTTTACGTAATAACGCT
AAAAGTTTTAGATGTGCTTTACTAAGTCATCGCGATGGAGCAAAAGTACA
TTTAGGTACACGGCCTACAGAAAAACAGTATGAAACTCTCGAAAATCAAT
TAGCCTTTTTATGCCAACAAGGTTTTTCACTAGAGAATGCATTATATGCA
CTCAGCGCTGTGGGGCATTTTACTTTAGGTTGCGTATTGGAAGATCAAGA
GCATCAAGTCGCTAAAGAAGAAAGGGAAACACCTACTACTGATAGTATGC
CGCCATTATTACGACAAGCTATCGAATTATTTGATCACCAAGGTGCAGAG
CCAGCCTTCTTATTCGGCCTTGAATTGATCATATGCGGATTAGAAAAACA
ACTTAAATGTGAAAGTGGGTCCGCGTACAGCGGATCCCGGGAATTCAGAT
CTTATTAAGCGGCCGCATAGATAACTGATCCAGTGTGCTGGAATTAATTC
GCTGTCTGCGAGGGCCAGCTGTTGGGGTGAGTACTCCCTCTCAAAAGCGG
GCATGACTTCTGCGCTAAGATTGTCAGTTTCCAAAAACGAGGAGGATTTG
ATATTCACCTGGCCCGCGGTGATGCCITTGAGGGTGGCCGCGTCCATCTG
GTCAGAAAAGACAATCTTTTTGTTGTCAAGCTTGAGGTGTGGCAGGCTTG
AGATCTGGCCATACACTTGAGTGACAATGACATCCACTTTGCCTTTCTCT
CCACAGGTGTCCACTCCCAGGTCCAACTGCAGGTCGAGCATGCATCTAGG
GCGGCCAATTCCGCCCCTCTCCCTCCCCCCCCCCTAACGTTACTGGCCGA
AGCCGCTTGGAATAAGGCCGGTGTGCGTTTGTCTATATGTGATTTTCCAC
CATATTGCCGTCTTTTGGCAATGTGAGGGCCCGGAAACCTGGCCCTGTCT
TCTTGACGAGCATTCCTAGGGGTCTTTCCCCTCTCGCCAAAGGAATGCAA
GGTCTGTTGAATGTCGTGAAGGAAGCAGTTCCTCTGGAAGCTTCTTGAAG
ACAAACAACGTCTGTAGCGACCCTTTGCAGGCAGCGGAACCCCCCACCTG
GCGACAGGTGCCTCTGCGGCCAAAAGCCACGTGTATAAGATACACCTGCA
AAGGCGGCACAACCCCAGTGCCACGTTGTGAGTTGGATAGTTGTGGAAAG
AGTCAAATGGCTCTCCTCAAGCGTATTCAACAAGGGGCTGAAGGATGCCC
AGAAGGTACCCCATTGTATGGGATCTGATCTGGGGCCTCGGTGCACATGC
TTTACATGTGTTTAGTCGAGGTTAAAAAAACGTCTAGGCCCCCCGAACCA
CGGGGACGTGGTTTTCCTTTGAAAAACACGATGATAAGCTTGCCACAACC
CACAAGGAGACGACCTTCCATGACCGAGTACAAGCCCACGGTGCGCCTCG
CCACCCGCGACGACGTCCCCCGGGCCGTACGCACCCTCGCCGCCGCGTTC
GCCGACTACCCCGCCACGCGCCACACCGTCGACCCGGACCGCCACATCGA
GCGGGTCACCGAGCTGCAAGAACTCTTCCTCACGCGCGTCGGGCTCGACA
TCGGCAAGGTGTGGGTCGCGGACGACGGCGCCGCGGTGGCGGTCTGGACC
ACGCCGGAGAGCGTCGAAGCGGGGGCGGTGTTCGCCGAGATCGGCCCGCG
CATGGCCGAGTTGAGCGGTTCCCGGCTGGCCGCGCAGCAACAGATGGAAG
GCCTCCTGGCGCCGCACCGGCCCAAGGAGCCCGCGTGGTTCCTGGCCACC
GTCGGCGTCTCGCCCGACCACCAGGGCAAGGGTCTGGGCAGCGCCGTCGT
GCTCCCCGGAGTGGAGGCGGCCGAGCGCGCCGGGGTGCCCGCCTTCCTGG
AGACCTCCGCGCCCCGCAACCTCCCCTTCTACGAGCGGCTCGGCTTCACC
GTCACCGCCGACGTCGAGTGCCCGAAGGACCGCGCGACCTGGTGCATGAC
CCGCAAGCCCGGTGCCTGACGCCCGCCCCACGACCCGCAGCGCCCGACCG
AAAGGAGCGCACGACCCCATGGCTCCGACCGAAGCCGACCCGGGCGGCCC
CGCCGACCCCGCACCCGCCCCCGAGGCCCACCGACTCTAGAGCTCGCTGA
TCAGCCTCGACTGTGCCTTCTAGTTGCCAGCCATCTGTTGTTTGCCCCTC
CCCCGTGCCTTCCTTGACCCTGGAAGGTGCCACTCCCACTGTCCTTTCCT
AATAAAATGAGGAAATTGCATCGCATTGTCTGAGTAGGTGTCATTCTATT
CTGGGGGGTGGGGTGGGGCAGGACAGCAAGGGGGAGGATTGGGAAGACAA
TAGCAGGCATGCTGGGGATGCGGTGGGCTCTATGGCTTCTGAGGCGGAAA
GAACCAGCTGGGGCTCGAGTGCATTCTAGTTGTGGTTTGTCCAAACTCAT
CAATGTATCTTATCATGTCTGTATACCGTCGACCTCTAGCTAGAGCTTGG
CGTAATCATGGTCATAGCTGTTTCCTGTGTGAAATTGTTATCCGCTCACA
ATTCCACACAACATACGAGCCGGAAGCATAAAGTGTAAAGCCTGGGGTGC
CTAATGAGTGAGCTAACTCACATTAATTGCGTTGCGCTCACTGCCCGCTT
TCCAGTCGGGAAACCTGTCGTGCCAGCTGCATTAATGAATCGGCCAACGC
GCGGGGAGAGGCGGTTTGCGTATTGGGCGCTCTTCCGCTTCCTCGCTCAC
TGACTCGCTGCGCTCGGTCGTTCGGCTGCGGCGAGCGGTATCAGCTCACT
CAAAGGCGGTAATACGGTTATCCACAGAATCAGGGGATAACGCAGGAAAG
AACATGTGAGCAAAAGGCCAGCAAAAGGCCAGGAACCGTAAAAAGGCCGC
GTTGCTGGCGTTTTTCCATAGGCTCCGCCCCCCTGACGAGCATCACAAAA
ATCGACGCTCAAGTCAGAGGTGGCGAAACCCGACAGGACTATAAAGATAC
CAGGCGTTTCCCCCTGGAAGCTCCCTCGTGCGCTCTCCTGTTCCGACCCT
GCCGCTTACCGGATACCTGTCCGCCTTTCTCCCTTCGGGAAGCGTGGCGC
TTTCTCAATGCTCACGCTGTAGGTATCTCAGTTCGGTGTAGGTCGTTCGC
TCCAAGCTGGGCTGTGTGCACGAACCCCCCGTTCAGCCCGACCGCTGCGC
CTTATCCGGTAACTATCGTCTTGAGTCCAACCCGGTAAGACACGACTTAT
CGCCACTGGCAGCAGCCACTGGTAACAGGATTAGCAGAGCGAGGTATGTA
GGCGGTGCTACAGAGTTCTTGAAGTGGTGGCCTAACTACGGCTACACTAG
AAGGACAGTATTTGGTATCTGCGCTCTGCTGAAGCCAGTTACCTTCGGAA
AAAGAGTTGGTAGCTCTTGATCCGGCAAACAAACCACCGCTGGTAGCGGT
GGTTTTTTTGTTTGCAAGCAGCAGATTACGCGCAGAAAAAAAGGATCTCA
AGAAGATCCTTTGATCTTTTCTACGGGGTCTGACGCTCAGTGGAACGAAA
ACTCACGTTAAGGGATTTTGGTCATGAGATTATCAAAAAGGATCTTCACC
TAGATCCTTTTAAATTAAAAATGAAGTTTTAAATCAATCTAAAGTATATA
TGAGTAAACTTGGTCTGACAGTTACCAATGCTTAATCAGTGAGGCACCTA
TCTCAGCGATCTGTCTATTTCGTTCATCCATAGTTGCCTGACTCCCCGTC
GTGTAGATAACTACGATACGGGAGGGCTTACCATCTGGCCCCAGTGCTGC
AATGATACCGCGAGACCCACGCTCACCGGCTCCAGATTTATCAGCAATAA
ACCAGCCAGCCGGAAGGGCCGAGCGCAGAAGTGGTCCTGCAACTTTATCC
GCCTCCATCCAGTCTATTAATTGTTGCCGGGAAGCTAGAGTAAGTAGTTC
GCCAGTTAATAGTTTGCGCAACGTTGTTGCCATTGCTACAGGCATCGTGG
TGTCACGCTCGTCGTTTGGTATGGCTTCATTCAGCTCCGGTTCCCAACGA
TCAAGGCGAGTTACATGATCCCCCATGTTGTGCAAAAAAGCGGTTAGCTC
CTTCGGTCCTCCGATCGTTGTCAGAAGTAAGTTGGCCGCAGTGTTATCAC
TCATGGTTATGGCAGCACTGCATAATTCTCTTACTGTCATGCCATCCGTA
AGATGCTTTTCTGTGACTGGTGAGTACTCAACCAAGTCATTCTGAGAATA
GTGTATGCGGCGACCGAGTTGCTCTTGCCCGGCGTCAATACGGGATAATA
CCGCGCCACATAGCAGAACTTTAAAAGTGCTCATCATTGGAAAACGTTCT
TCGGGGCGAAAACTCTCAAGGATCTTACCGCTGTTGAGATCCAGTTCGAT
GTAACCCACTCGTGCACCCAACTGATCTTCAGCATCTTTTACTTTCACCA
GCGTTTCTGGGTGAGCAAAAACAGGAAGGCAAAATGCCGCAAAAAAGGGA
ATAAGGGCGACACGGAAATGTTGAATACTCATACTCTTCCTTTTTCAATA
TTATTGAAGCATTTATCAGGGTTATTGTCTCATGAGCGGATACATATTTG
AATGTATTTAGAAAAATAAACAAATAGGGGTTCCGCGCACATTTCCCCGA
AAAGTGCCACCTGACGTC

Subsequently pIRESpuro2/TR was transfected into L6-GLUT4myc cells. Clones stably expressing the regulatory plasmid were selected with 0.5 μg/ml puromycin (InvivoGen).

L6-GLUT4myc cells containing AS160-like were grown in MEMα (PAN) supplemented with 10% fetal calf serum (FCS) (PAA, tet-free), 2 μg/ml blasticidin (Calbiochem), 0.5 μg/ml puromycin (InvivoGen), 200 μg/ml hygromycin (Invitrogen). Functionality of the tet-repressor was controlled with a tetracycline-inducible GFP expression plasmid (pCDNA5/TO-GFP). In the absence of tetracycline (or doxycyclin) only a small number of cells express GFP. Addition of doxycyclin increases the number of GFP expressing cells about 10 fold (data not shown).

To obtain a tet-inducible expression of the isoform2 of AS160, the pCDNA5 vector from Invitrogen containing the gene of isoforms 2 of AS160 was used. Selection of stable clones was performed with hygromycin (200 μg/ml, Invitrogen). Expression of isoform2 of AS160 was examined via western blot analysis with an AS160-specific antibody recognizing full-length AS160 and isoform2 of AS160. Expression of isoform2 of AS160 was induced with 1 μg/ml doxycyclin (Sigma). Functionality of the tet-repressor was investigated with addition of doxycyclin (1 μg/ml, Sigma) and subsequent western blot analysis.

For all examples L6-GLUT4myc cells containing isoform2 of AS160 were grown in MEMα (PAN) supplemented with 10% fetal calf serum (FCS) (PAA, tet-free), 2 μg/ml blasticidin (Calbiochem), 0.5 μg/ml puromycin (InvivoGen), 200 μg/ml hygromycin (Invitrogen). Expression of isoform2 of AS160 was induced with 1 μg/ml doxycyclin (Sigma). L6-wildtype (wt) (ATCC: CRL-1458) cells were grown in MEMα+GlutaMax (Gibco) supplemented with 10% FCS (PAA, tet-free) and 1% penicillin/streptomycin (PAA). L6-GLUT4myc cells were grown in MEMα+GlutaMax (Gibco) supplemented with 10% FCS (PAA, tet-free), 1% penicillin/streptomycin (PAA) and 2 μg/ml blasticidin (Calbiochem). All cells were grown at 37° C. and 5% CO₂. L6-GLUT4myc cells containing isoform2 of AS160 were incubated in starve medium (MEMα) 3-4 hours prior to each experiment.

For Western blot analysis proteins were separated on SDS-PAGE gels (4-12% resolving gel, Invitrogen), transferred to PVDF membranes (Roche) and blocked with Roti-Block® (Roth) for 1 hour. Membranes were incubated with primary antibodies overnight. The anti-AS160 antibody was from Upstate. Membranes were washed in TBST and incubated with the appropriate secondary horseradish peroxidase conjugated antibody (Santa Cruz). Immunoreactive bands were visualized with LumiLight (Roche) and detected with Lumi-lmager (Böhringer Ingelheim).

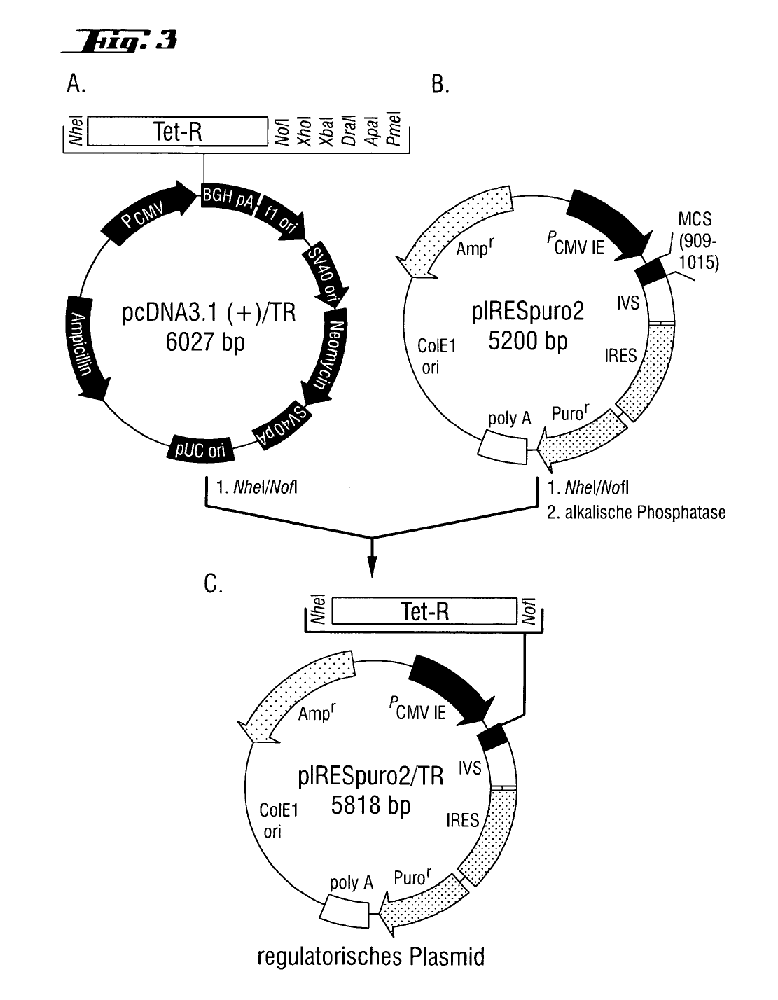

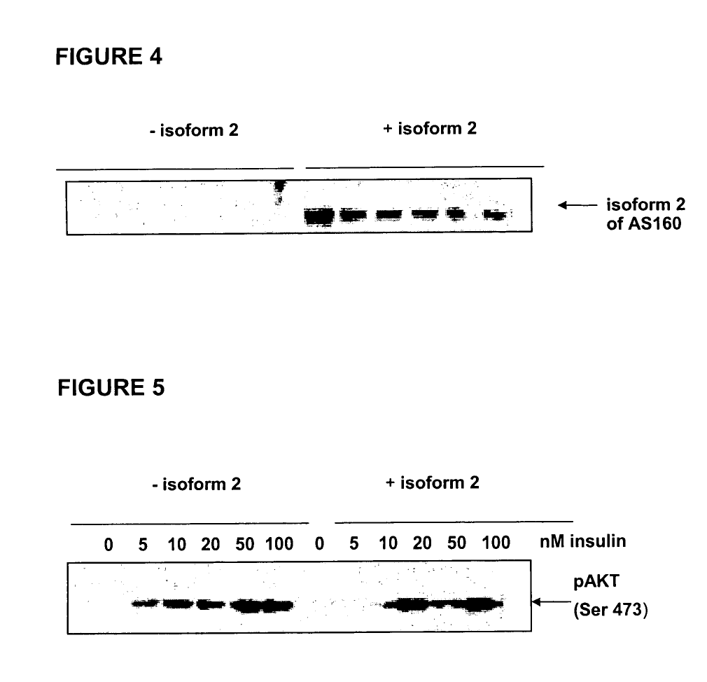

Analysis of the functionality of the tet-repressor revealed that isoform2 of AS160 is expressed only in the presence of doxycycline (FIG. 4).

In order to examine tetracycline- and insulin-dependent expression of isoform2 of AS160 protein in L6-GLUT4myc cells containing AS160-like, a western blot analysis was performed. The expression of AS160-like protein was induced with 1 μg/ml doxycyclin for 48 hours as described above. Subsequently, the cells were stimulated with insulin (5 nM to 50 nM; Sanofi-Aventis) for 20 minutes. Cell extracts were prepared, separated via SDS-PAGE and transferred to a PVDF membrane as described above.

FIG. 4 shows a representative western blot of doxycyclin-inducible expression of AS160-like protein in L6-GLUT4myc cells containing the AS160-like transcript. Incubation with insulin induced the phosphorylation of AKT (Ser 473) (FIG. 5). Expression of the isoform2 of AS160 had no effect on the activation of AKT.

### Example 3

### Phosphorylation of AKT and AS160

To study phosphorylation of AKT and AS160 the cells described in Example 2 were used. AS160 is activated by phosphorylation on critical motifs (RXRXXS/T). Known phospho-sites in AS160 are Ser 570, Ser 588, Thr 642 and Thr 751 (Sano et al., 2003). One of the kinases responsible for the activation of AS160 is AKT (Kane et al., 2002). In order to determine the activation status of the isoform2 of AS160 the In-cell western blot technique was used. This method allows the detection of specific proteins directly in 96 well plates without preparation of cell extracts. The specific antibody used in this assay recognizes the phosphorylated Thr 642 phosphorylation site of AS160 and AS160-like protein.

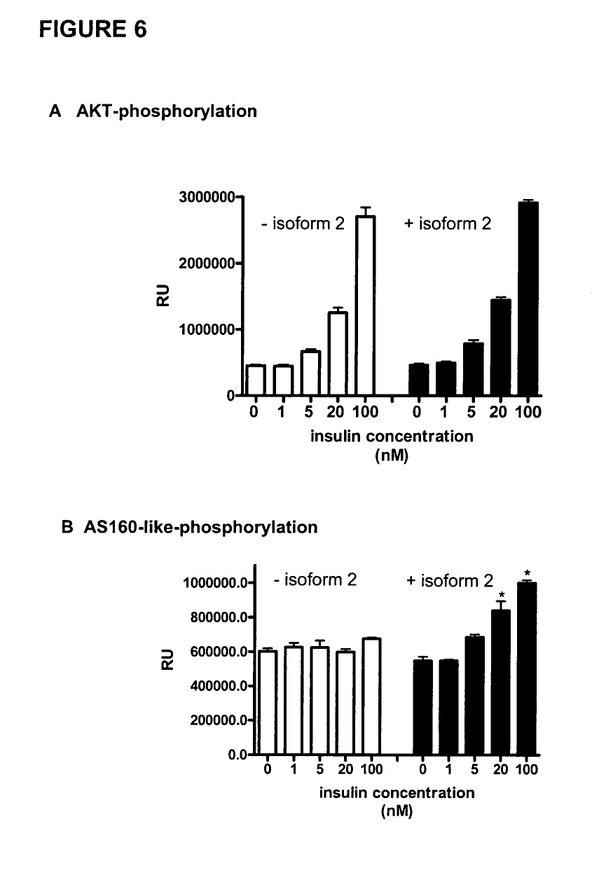

For this, cells were seeded into 96-well plates (black, Nunc) and grown for 48 hours. Cells were starved for 3-4 hours with MEMα (PAN) containing 2% horse serum (Cambrex). After removal of medium cells were fixed in 3.7% freshly prepared para-formaldehyde (Sigma) for 20 minutes. Cells were permeabilized with PBS+0.1% Triton-X-100. Blocking was performed with Odyssey blocking buffer (Licor) overnight at 4° C. Primary antibodies were incubated for 2 hours at room temperature. The anti-phosphoAKT (Ser 473, Cat No.: 44-621 G) and the anti-phosphoAS160 (Thr 642, Cat No.: 44-1071 G) were from Biosource. After incubation of the primary antibody, cells were washed with PBS+0.1% Tween20. The secondary anti-rabbit-IgG-800-CW antibody (Rockland Cat No.: 611-131-122) was incubated for 1 hour. For detection of DNA TO-PRO3 dye (Molecular Probes, Cat No.: T3605) was used. Fluorescence signals (FIG. 6) are presented as relative units (RU).

As a control the dose-dependent phosphorylation/activation of AKT on Ser 473 was determined in the same experimental setting. Insulin induces AKT phosphorylation and phosphorylation of full-length or isoform2 of AS160 in a dose-dependent manner (FIGS. 6A and B). No phosphorylation of AS160 or isoform2 of AS160 protein was observed in cells without doxycyclin-induced expression of AS160-like protein.

### Example 4

### Effect of Isoform2 of AS160 Protein on Glucose Uptake

To examine glucose uptake of the cells, the respective cells were plated in 96 well Cytostar-T scintillating microplates (Amersham). After 48 hours cells were serum-starved (3-4 hours) and treated with inhibitors as indicated. Uptake of 2-deoxyglucose (0.01 MBq per well, Amersham) was performed as already described (Voss et al., 2005). Nonspecific uptake was determined in the presence of 40 μM cytochalasin B (Calbiochem). This value was subtracted from all other values. Measurement occurred in a Wallac Microbeta counter (Perkin Elmer). Uptake of 2-deoxyglucose is presented as counts per million (cpm).

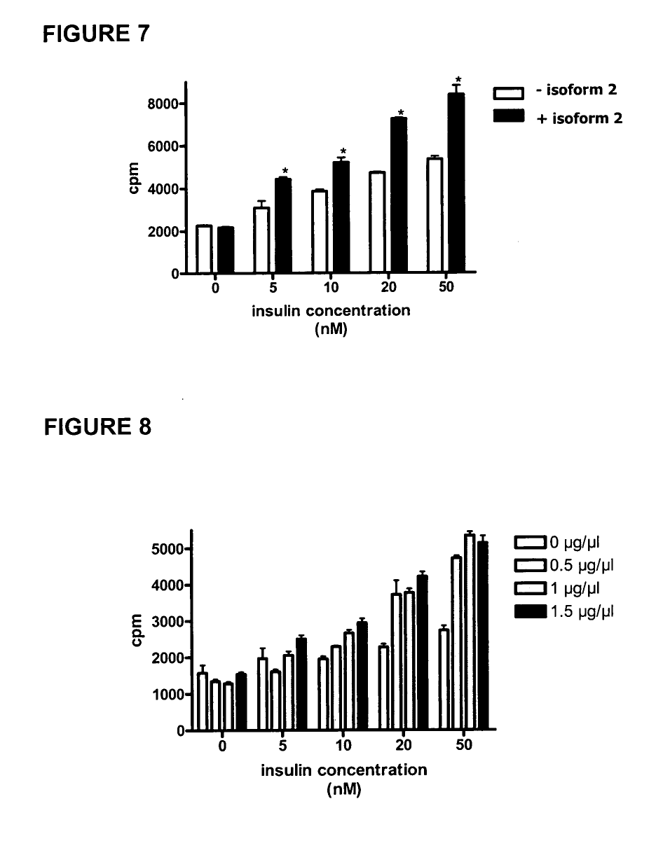

The uptake of 2-deoxyglucose in L6-GLUT4myc cells expressing isoform 2 of AS160 was examined in response to insulin (FIG. 7). The data show that expression of isoform2 of AS160 increases the uptake of glucose up to 4 fold after stimulation with insulin (concentration of 50 nM insulin). Without expression of isoform 2 of AS160 insulin induces glucose uptake up to a maximum of 2 fold.

The increase of glucose uptake in these cells was induced by doxycyclin in a dose-dependent manner (FIG. 8) which correlated with isoform2 of AS160 protein levels.

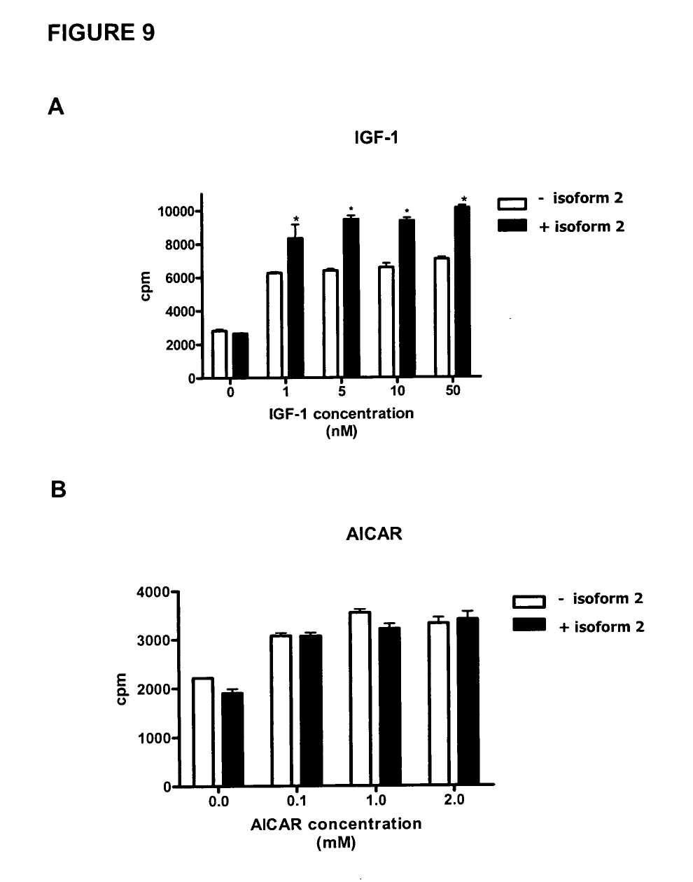

From the literature it is already known that IGF-1 (insulin like growth factor-1, R&D Systems, Cat No.: 291-G1) and the AMPK (5′-AMP-activated protein kinase) activator AICAR (5-Aminoimidazole-4-carboxaide 1-beta-D-ribofuranoside, Biomol Cat No.: El-330) also stimulate glucose uptake in skeletal muscle cells (Ciaraldi et al., 2002). Therefore, we examined the effects of the expression of isoform 2 of AS160 on IGF-1 and AICAR stimulated glucose uptake (FIG. 9). In the absence of isoform 2 of AS160, compared to insulin the uptake of glucose in cells stimulated with IGF-1 is higher (3 fold) than after stimulation with insulin (2 fold) (FIG. 9A).

The uptake of glucose in cells stimulated with AICAR is lower (1.5 fold) than the uptake of cells stimulated with insulin (FIG. 9B). Expression of isoform 2 of AS160 protein further increased the uptake of glucose after stimulation with IGF-1 (up to 5 fold), whereas no additional effect of expression of isoform 2 of AS160 was observed after stimulation with AICAR. These data indicate that stimulation of glucose uptake can occur via AMPK in a manner independent of isoform 2 of AS160 and via AKT in an isoform2-dependent manner. Thereby the cell system can be used to differentiate between isoform2-dependent and -independent effects.

### Example 5

### Effect of Metformin on Glucose Uptake

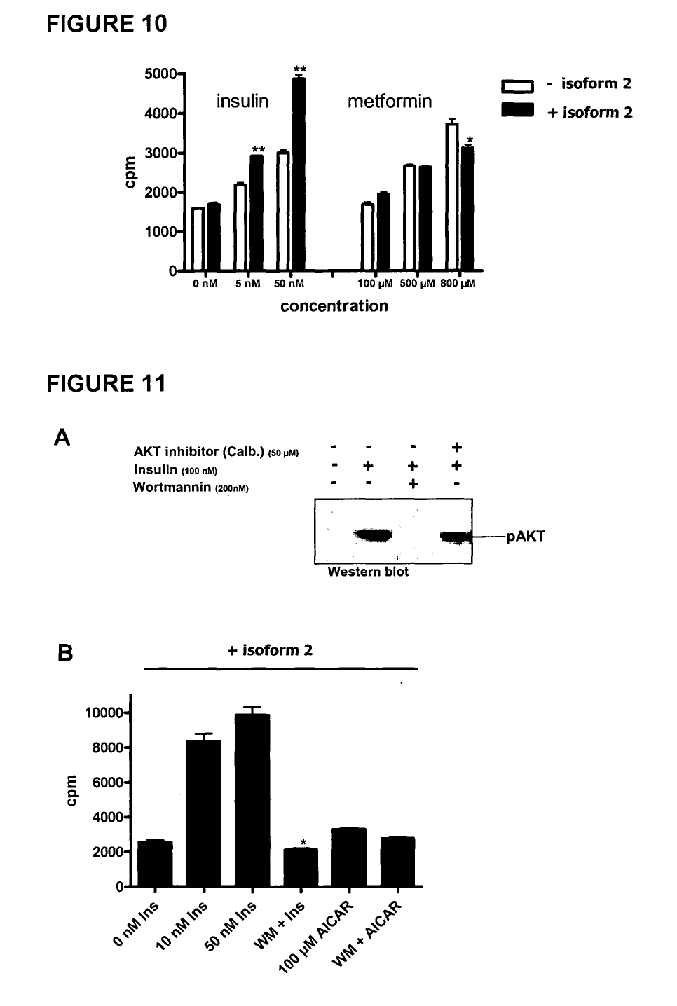

The glucose-lowering effects of metformin (dimethylbiguanide) in type 2 diabetes are already well documented (Karlsson et al., 2005a); however, its exact mechanism of action is uncertain despite its known therapeutic benefits. We examined the effect of isoform 2 of AS160 protein on glucose uptake after stimulation with different concentrations of metformin, wherein the test was carried out as described above (FIG. 10). In comparison to insulin, the expression of isoform2 of AS160 protein has no increasing effect on glucose uptake. This effect seems to be comparable with the effect observed after stimulation with AICAR. These data suggest that metformin-induced effects are also independent from isoform 2 of AS160 protein and might be mediated by AMPK.

### Example 6

### Effect of AKT Inhibitors

To obtain a more detailed analysis of the signaling cascade leading to the activation of isoform 2 of AS160, two different inhibitors were tested. Wortmannin (Upstate Cat No.: 12-338) is an already well established compound known to inhibit at the level of the PI3-kinase (PI3K) and subsequently leading to a reduced phosphorylation of AKT, which signals downstream of PI3-kinase (Okada et al., 1994). The second compound used is 1 L-6-hydroxymethyl-chiro-inositol 2-(R)-2-O-methyl-3-O-octadecylcarbonate abbreviated with AKT inhibitor Calb.; Calbiochem Cat No.: 124005). This compound is described as a selective inhibitor of AKT only weakly interfering with PI3K (Hu et al., 2000).

As expected, Western blot analysis revealed that wortmannin completely abrogated the phosphorylation of AKT (Ser 473) (FIG. 11A). The AKT inhibitor Calb. had no effect on the phosphorylation of AKT (Ser 473) (FIG. 11A). To examine the efficacy of the AKT inhibitor Calb., an analysis of signaling molecules downstream of AKT is required, because the phosphorylation of AKT might not directly correlate with the activity of AKT. Phosphorylation of AKT on Ser 473 was detected with a phospho-specific antibody from Biosource (Cat No.: 44-621G).

Wortmannin was also able to completely abrogate the uptake of glucose of L6-GLUT4myc cells expressing isoform 2 of AS160, whereas glucose uptake of AICAR stimulated cells remained nearly unchanged (FIG. 11B). So far, the process of glucose uptake appears to be mediated by the PI3K-AKT signaling pathway.

### Example 7

### Time-Dependency of Glucose Uptake of L6-GLUT4-myc Cells Expressing Isoform2 of AS160

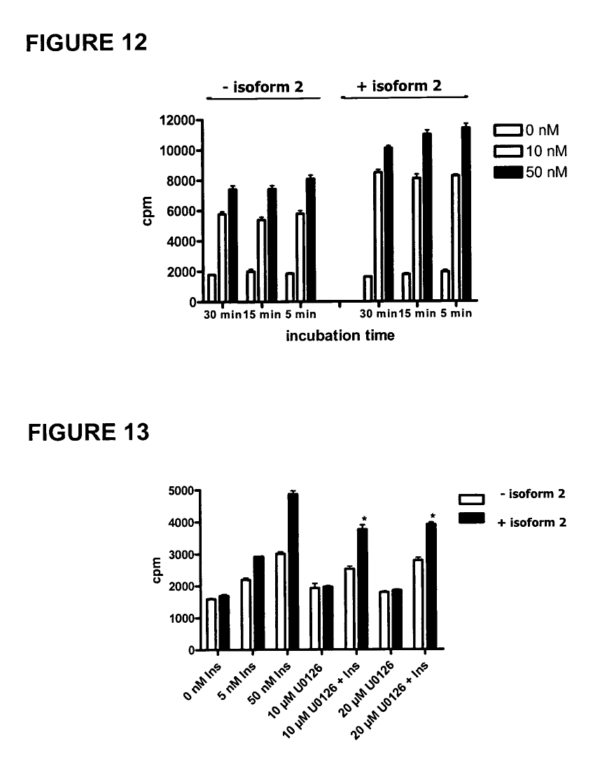

The contribution of isoform2 of AS160 responsible for improved glucose uptake might be the acceleration of the translocation of GLUT4 to the plasma membrane, a higher overall amount of GLUT4 protein at the membrane or an impaired endocytosis. In order to elucidate this mechanism a time-dependent analysis of glucose uptake in cells expressing isoform2 of AS160 with or without induction of isoform2 of AS160 was performed. Glucose uptake at 3 different time-points was investigated (5, 15, 30 minutes). Cells were stimulated with 2 different insulin concentrations (10 nM, 50 nM). FIG. 12 shows that the maximum of glucose uptake is already observed after 5 minutes stimulation with 50 nM insulin. A difference between L6-GLUT4myc cells expressing isoform2 of AS160 and cells without isoform2 of AS160 expression could not be detected. These data suggest that isoform 2 of AS160 does not accelerate glucose uptake within the examined time points.

Another potential reason for the enhanced uptake of glucose in the presence of isoform2 of AS160 might be an increased amount of GLUT4 at the plasma membrane. Translocation of GLUT4 is a key event in the induction of glucose uptake.

### Example 8

### Effect of MEKK/ERK Inhibitor U0126

To study the role of mitogen-activated kinases (MAPK) in the negative regulation of insulin-signaling as well as a contribution to insulin-resistance in skeletal muscle cells, we examined the effect of a commonly used MEKK/ERK inhibitor U0126 (Upstate Cat No.: 19-147; DeSilva et al., 1998) on glucose-uptake. Incubation of cells with the MEKK/ERK inhibitor U0126 (10 μM, 20 μM) revealed a significantly improved uptake of glucose after additional stimulation with 5 nM insulin. This effect mainly occurred in cells expressing isoform2 of AS160 (FIG. 13).

This finding indicates that the MEKK/ERK kinases also negatively influence insulin signaling and glucose uptake in an isoform2-dependent manner in the cell model used. Similar data were recently published by the group of J. Zierath (Bouzakri and Zierath, 2007) for a TNF-α induced insulin-resistance cell model, which speculate that silencing of especially MAPK4 could be a novel approach to restore appropriate insulin signaling in skeletal muscle cells.

### Example 9

### Establishment of an Insulin-Resistance Cell Model

As in type II diabetes peripheral insulin-resistance becomes immanent, we aimed to establish a cell-based insulin-resistance model that allows studying the molecular basis of insulin-resistance and in parallel allows developing strategies to restore insulin-sensitivity. Cell-culture based models of insulin-resistance are already well established for adipocytes (Nelson et al., 2002; Greene et al., 2001) and L6 myotubes (Walgren et al., 2003).

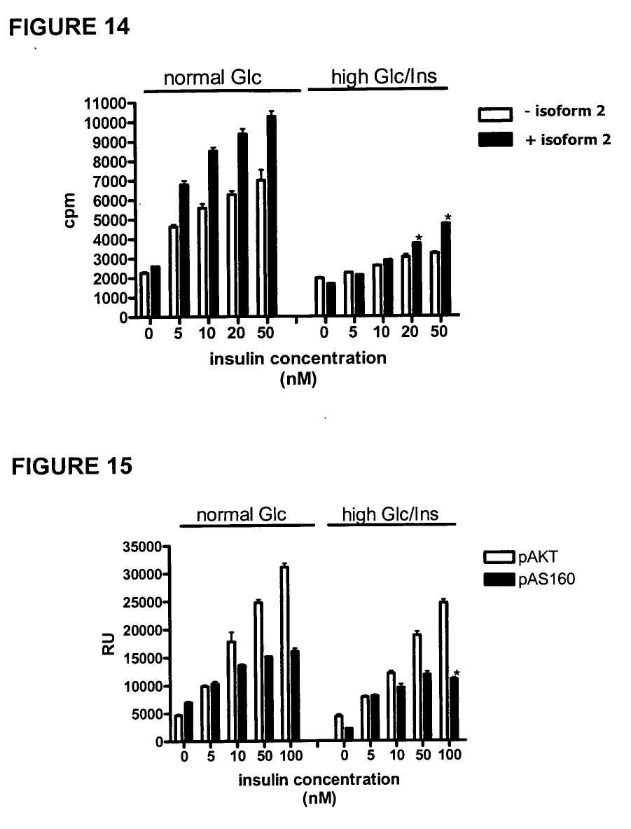

In this model, cells were grown under high glucose/insulin conditions (25 mM glucose+10 nM insulin) overnight to induce insulin resistance (Walgren et al., 2003). Compared to cells grown under normal glucose conditions (FIG. 14, left), glucose uptake of cells treated with high glucose and insulin, is markedly reduced (FIG. 14, right). Expression of isoform 2 of AS160 slightly improves glucose uptake but is not able to restore insulin sensitivity.

In-cell western blot analysis revealed that also under high glucose plus insulin conditions AKT (Ser 473, Biosource Cat No.: 44-621G) and AS160 (Thr 642, Biosource Cat No.: 44-1071G) are still phosphorylated in a dose-dependent manner (FIG. 15). However the phosphorylation signal is decreased to 72% compared to untreated conditions (normal Glc).

In addition, the effect of metformin under high glucose conditions was examined. For this purpose metformin (800 μM) was incubated overnight under normal conditions and in parallel under high glucose plus insulin conditions (25 mM glucose+10 nM insulin).

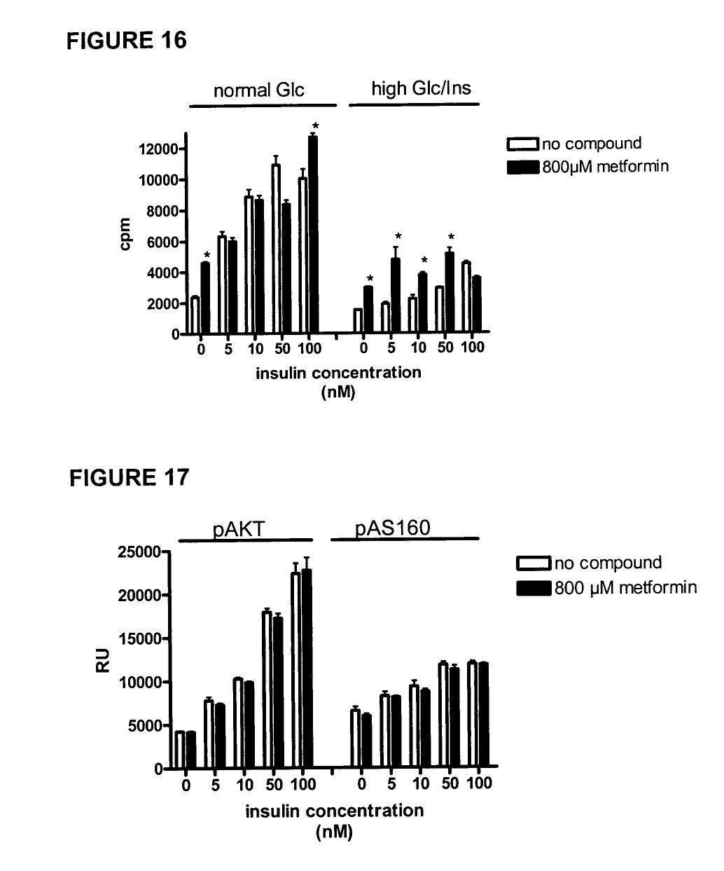

Isoform2-expression was induced in all wells. FIG. 16 shows that metformin significantly enhanced basal glucose uptake under normal conditions. Additional stimulation with different insulin concentrations with exception of 100 nM insulin did not further improve glucose uptake under normal conditions, indicating that metformin has no sensitizing effect.

Under high glucose plus insulin conditions metformin significantly increased basal glucose uptake as well as glucose uptake after stimulation with 5, 10 and 50 nM insulin (FIG. 16, right). However, the maximum of glucose uptake under insulin-resistant conditions does not exceed the basal levels after metformin stimulation, demonstrating again that metformin action in muscle is independent from insulin-stimulated glucose disposal.

Parallel examination of the phosphorylation status of AKT and AS160 under normal and insulin-resistant conditions revealed that treatment of cells with metformin has no effect on activation of AKT or AS160 (FIG. 17). These data indicate that metformin mediated effects do not depend on AKT or AS160 activation.

### Example 10

### Examination of GLUT4 Translocation

Although the cell-based glucose uptake is very reproducible and allows for a highly sensitive screening of compounds, it might still be improved for a high throughput screening (HTS). One of the rate-limiting steps in this context is the usage of radioactively labeled glucose in the uptake experiments. To provide an improved basis for this assay in HTS screening, we examined the correlation between increased glucose uptake and translocation of GLUT4 to the plasma membrane.

For this purpose, a technique suitable for screening of compounds was applied. Laser-scanning fluorescence microplate cytometry (ACUMEN technique, LIT) allows the distinct multiparametric analysis of single fluorescent cells in microplates (Bowen and Wylie, 2006). For laser-scanning fluorescence microplate cytometer (Acumen) cells were plated in 96 well plates (Biocat, black). Serum-starved cells were treated as indicated. Briefly, cells were fixed in 3.75% para-formaldehyde (Sigma) for 20 minutes. Subsequently, quenching occurred with 100 nM NH₄Cl. Cells were blocked with an adequate blocking solutions for a minimum of 1 hour. Primary antibody (monoclonal anti-myc 9E10, Santa Cruz, sc-40) was incubated for 1 hour. The secondary goat-anti mouse IgG (Alexa Fluor 488, Molecular Probes) was incubated in the presence of Sytox-orange for 1 hour (Bowen and Wylie, 2006).

Based on this highly sensitive procedure it is possible to screen various cell numbers for plasma-membrane based GLUT4-myc-display. Cells were counter-stained with a DNA dye (SytoxOrange) to confirm equal cell numbers.

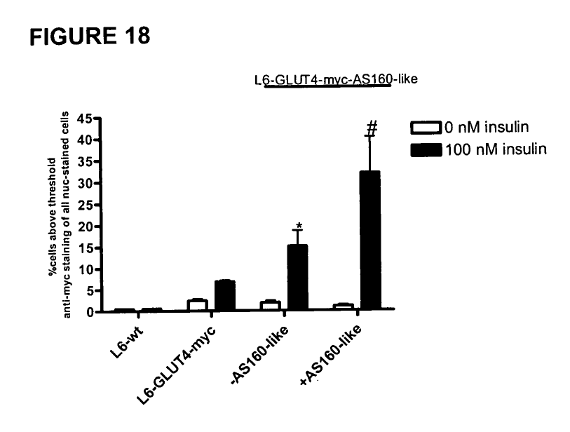

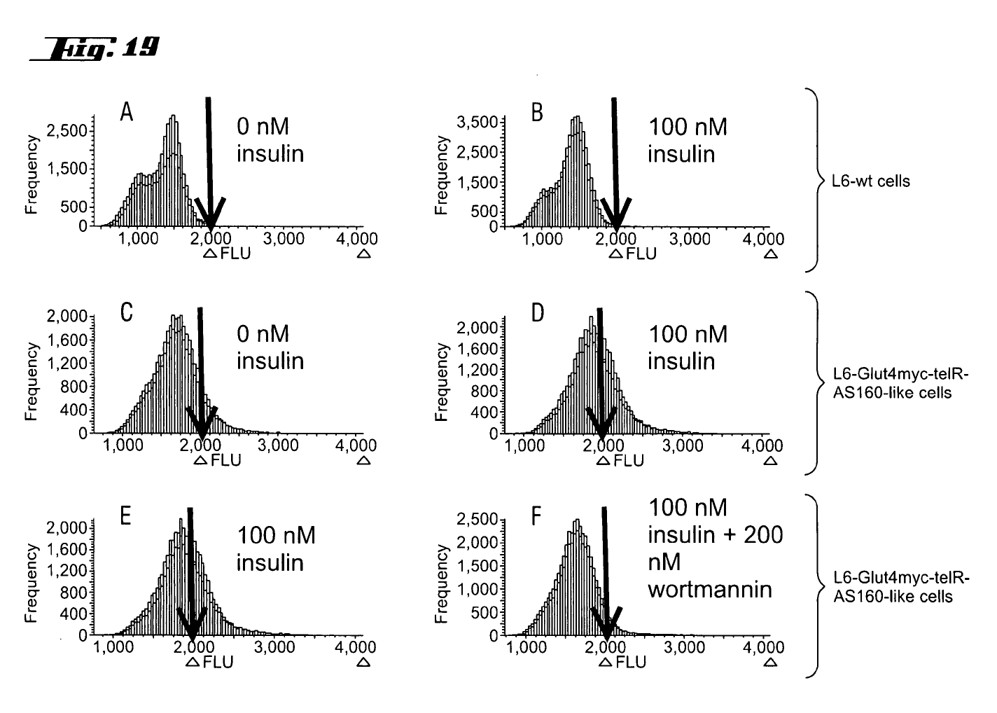

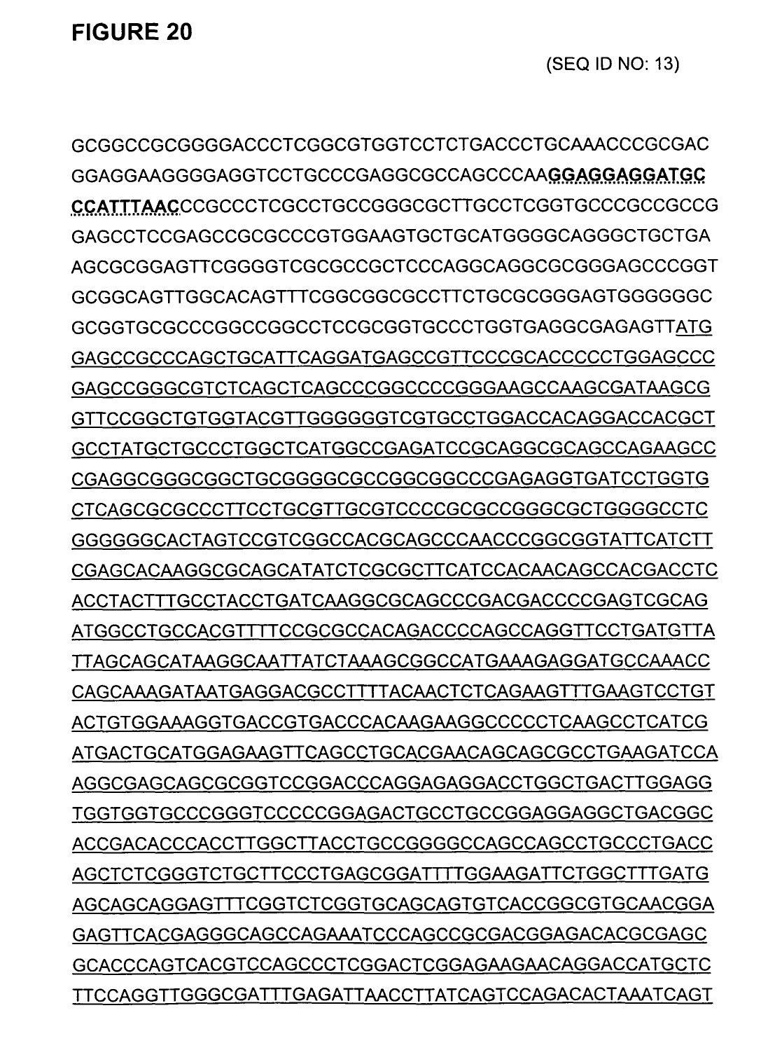

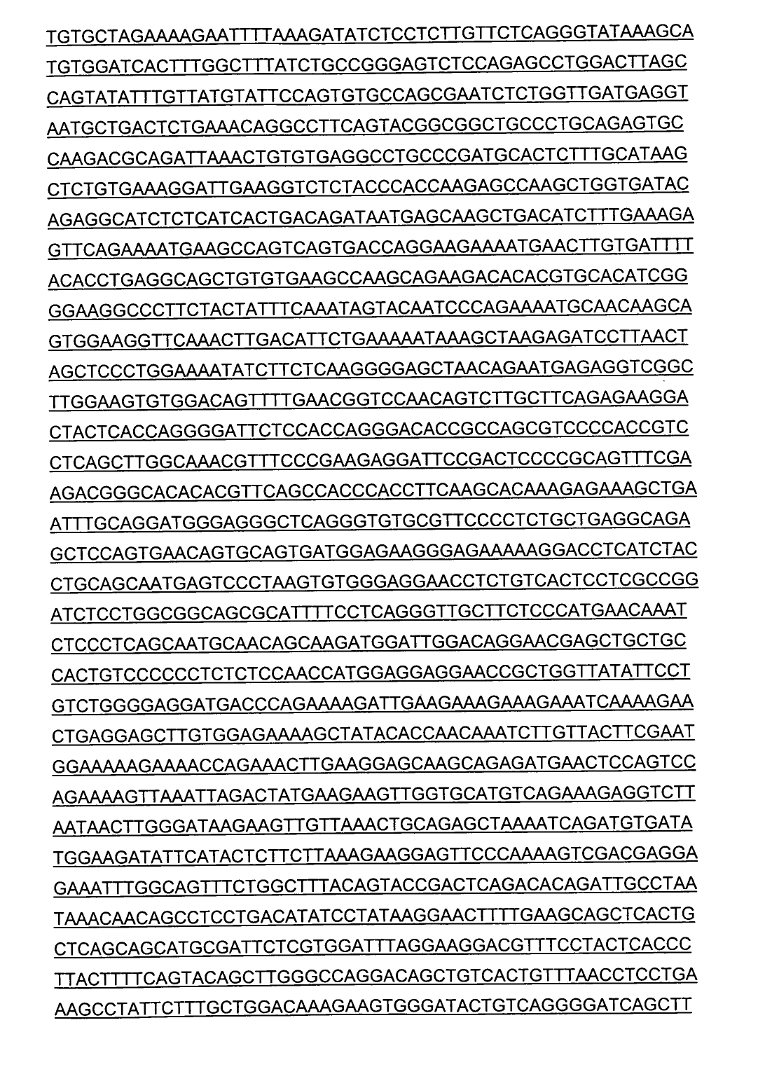

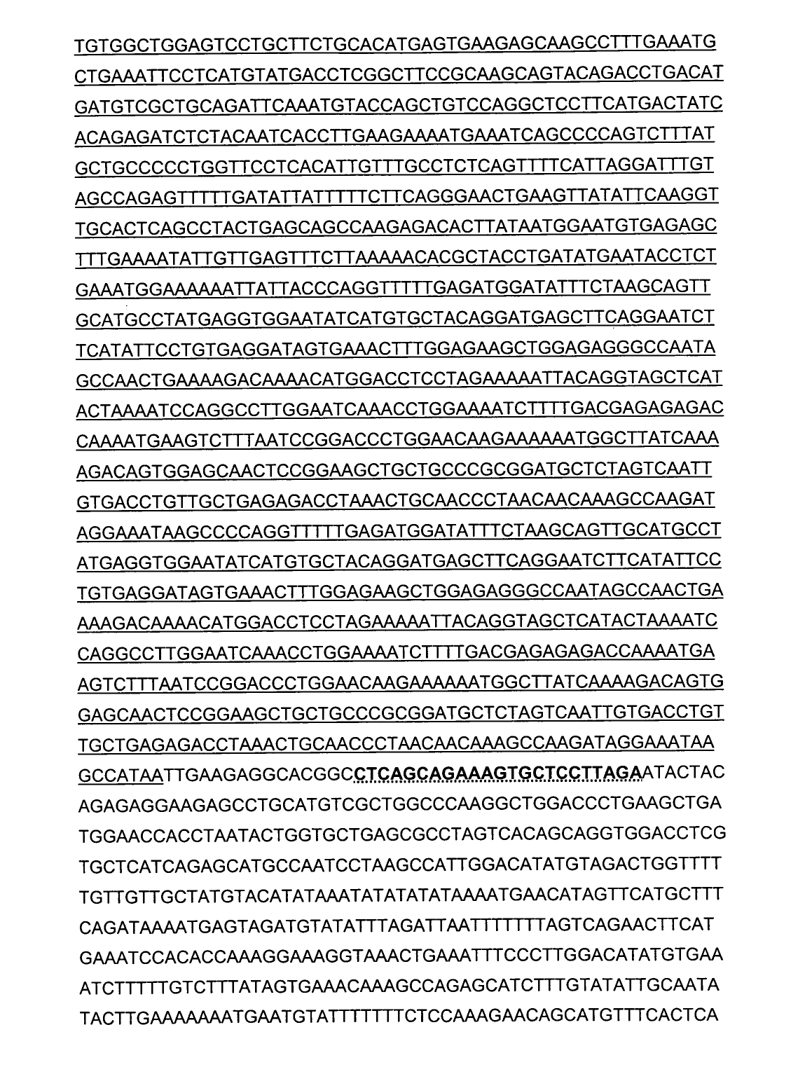

As an additional control, the parental cell lines, L6-wt and L6-GLUT4-myc were is included in this experiment. The ACUMEN experiment revealed a significantly increased translocation of GLUT4 to the plasma membrane (˜15%) (FIG. 18). As expected GLUT4 translocation is already slightly induced after stimulation with 100 nM insulin alone. Translocation is also increased in cells that contain the AS160-like expression cassette, but were not induced with doxycyclin. This phenomenon probably results from a leaky promoter.

A graphic presentation of GLUT4 translocation obtained with the laser-scanning fluorescence microplate cytometer (ACUMEN) is shown in FIG. 19. In these histograms the curves represents the total number of detected cells stained with a DNA marker (SytoxOrange). Cells left of the arrow shown in lighter grey do not display plasma-membrane bound GLUT4. Cells right of the arrow shown in lighter grey represent the population of cells that display the myc-tagged GLUT4 at the plasma-membrane.

### Example 11

### Localization of GLUT4 and Isoform2 of AS160

Immunofluorescence based analysis was applied to detect the localization of GLUT4-myc and AS160-like in the rat myoblast cell line L6-GLUT4-myc-tetR-AS160-like.

Cells were grown on sterile cover slides in 12 well plates. Expression of isoform2 of AS160 was induced with doxycyclin treatment for 48 hours. Serum-starved cells were treated with 100 nM insulin for 20 minutes or were left untreated. After stimulation, cells were fixed in 3.75% para-formaldehyde (Sigma) for 20 minutes. Subsequently, cells were permeabilised with 0.1% TritonX100 in PBS (Icon Biomedicals) or left unpermeabilised (as indicated) for 5 minutes at room temperature. The permeabilisation procedure allows the detection of intracellular localized compartments. Membrane-bound proteins are visualized without permeabilising the cells. Blocking of cells occurred for a minimum of 1 hour in PBS+1% BSA (USB). Primary antibodies (monoclonal anti-myc, Santa Cruz, sc-40 and polyclonal anti-AS160 Upstate) were incubated overnight. The secondary antibodies used are anti-rabbit Alexa488 to detect isoform2 of AS160 and anti-mouse A546 to detect GLUT4-myc. Incubation occurred is for 1 hour (dark). Cells were mounted in 15 μl Dako Cytomation Fluorescent Mounting Medium (DakoCytomation), examined by confocal laser-scanning microscopy with Leica DM IRE2 and analyzed with Leica DM SDK software.

Pictures obtained are shown in FIG. 21. The data indicate that isoform2 of AS160 and GLUT4-myc are both localized in the peri-nuclear compartment of cells under basal conditions. Insulin stimulation (100 nM) induces the translocation of GLUT4-myc to the plasma membrane. isoform2 of AS160 protein remains in the peri-nuclear compartment.

### REFERENCE LIST

Bouzakri, K. and Zierath, J. R. (2007). MAP4K4 gene silencing in human skeletal muscle prevents TNF-alpha -induced insulin resistance. J Biol Chem. 282, 7783-7789

Bowen, W. P. and Wylie, P. G. (2006). Application of laser-scanning fluorescence microplate cytometry in high content screening. Assay. Drug Dev. Technol. 4, 209-221.

Ciaraldi, T. P., Carter, L., Rehman, N., Mohideen, P., Mudaliar, S., and Henry, R. R. (2002). Insulin and insulin-like growth factor-1 action on human skeletal muscle: preferential effects of insulin-like growth factor-1 in type 2 diabetic subjects. Metabolism. 51, 1171-1179.

DeSilva, D. R., Jones, E. A., Favata, M. F., Jaffee, B. D., Magolda, R. L., Trzaskos, J. M., and Scherle, P. A. (1998). Inhibition of mitogen-activated protein kinase kinase blocks T cell proliferation but does not induce or prevent anergy. J Immunol. 160, 4175-4181.

Elbashir S. M., Harborth J., Lendeckel W., Yalcin A., Weber K., and Tuschl T. (2001). Duplexes of 21-nucleotides RNAs mediate RNA interference in mammalian cell culture. Nature. 411. 494-498

Greene, E. L., Nelson, B. A., Robinson, K. A., and Buse, M. G. (2001). alpha-Lipoic acid prevents the development of glucose-induced insulin resistance in 3T3-L1 adipocytes and accelerates the decline in immunoreactive insulin during cell incubation. Metabolism. 50, 1063-1069.

Hu, Y., Qiao, L., Wang, S., Rong, S. B., Meuillet, E. J., Berggren, M., Gallegos, A., Powis, G., and Kozikowski, A. P. (2000). 3-(Hydroxymethyl)-bearing phosphatidylinositol ether lipid analogues and carbonate surrogates block PI3-K, Akt, and cancer cell growth. J Med Chem. 43, 3045-3051.

Kane, S., Sano, H., Liu, S. C., Asara, J. M., Lane, W. S., Garner, C. C., and Lienhard, G. E. (2002). A method to identify serine kinase substrates. Akt phosphorylates a novel adipocyte protein with a Rab GTPase-activating protein (GAP) domain. J Biol. Chem. 277, 22115-22118.

Karlsson, H. K., Hallsten, K., Bjornholm, M., Tsuchida, H., Chibalin, A. V., Virtanen, K. A., Heinonen, O. J., Lonnqvist, F., Nuutila, P., and Zierath, J. R. (2005a). Effects of metformin and rosiglitazone treatment on insulin signaling and glucose uptake in patients with newly diagnosed type 2 diabetes: a randomized controlled study. Diabetes. 54, 1459-1467.

Karlsson, H. K., Zierath, J. R., Kane, S., Krook, A., Lienhard, G. E., and Wallberg-Henriksson, H. (2005b). Insulin-stimulated phosphorylation of the Akt substrate AS160 is impaired in skeletal muscle of type 2 diabetic subjects. Diabetes. 54, 1692-1697.

Nelson, B. A., Robinson, K. A., and Buse, M. G. (2002). Defective Akt activation is associated with glucose—but not glucosamine-induced insulin resistance. Am. J Physiol Endocrinol Metab. 282, E497-E506.

Okada, T., Kawano, Y., Sakakibara, T., Hazeki, O., and Ui, M. (1994). Essential role of phosphatidylinositol 3-kinase in insulin-induced glucose transport and antilipolysis in rat adipocytes. Studies with a selective inhibitor wortmannin. J Biol. Chem. 269, 3568-3573.

Sano, H., Kane, S., Sano, E., Miinea, C. P., Asara, J. M., Lane, W. S., Garner, C. W., and Lienhard, G. E. (2003). Insulin-stimulated phosphorylation of a Rab GTPase-activating protein regulates GLUT4 translocation. J Biol. Chem. 278, 14599-14602.

Tusch T., Zamore P D., Lehmann R., Bartel D P. and Sharp P A (1999). Targeted mRNA degradation by double-stranded RNA in vitro. Genes and Development 13. 3191-3197

Voss, M. D., Beha, A., Tennagels, N., Tschank, G., Herling, A. W., Quint, M., Gerl, M., Metz-Weidmann, C., Haun, G., and Korn, M. (2005). Gene expression profiling in skeletal muscle of Zucker diabetic fatty rats: implications for a role of stearoyl-CoA desaturase 1 in insulin resistance. Diabetologia. 48, 2622-2630.

Walgren, J. L., Vincent, T. S., Schey, K. L., and Buse, M. G. (2003). High glucose and insulin promote O-GlcNAc modification of proteins, including alpha-tubulin. Am. J Physiol Endocrinol Metab. 284, E424-E434.

Wang Q., Khayat Z., Kishi Y., Ebina Y., and Klip A. (1998). GLUT4 translocation by insulin in intact muscle cells: detection by a fast and quantitative assay. FEBS Lett. 427 (2), 193-197

Yuan, J. S., Reed, A., Chen, F., and Stewart, C. N., Jr. (2006). Statistical analysis of real-time PCR data. BMC Bioinformatics. 7:85, 85.

Zeigerer, A., McBrayer, M. K., and McGraw, T. E. (2004). Insulin stimulation of GLUT4 exocytosis, but not its inhibition of endocytosis, is dependent on RabGAP AS160. Mol. Biol. Cell. 15, 4406-4415.

If not indicated otherwise, standard laboratory methods were or can be performed according to standard procedures known in the art, e.g. as outlined the following laboratory standard literature:

Sambrook et al. (1989) Molecular Cloning: A Laboratory Manual. Second edition. Cold Spring Harbor Laboratory Press, Cold Spring Harbor, N.Y. 545 pp;

Current Protocols in Molecular Biology; regularly updated, e.g. Volume 2000; Wiley & Sons, Inc; Editors: Fred M. Ausubel, Roger Brent, Robert Eg. Kingston, David D. Moore, J. G. Seidman, John A. Smith, Kevin Struhl.

Current Protocols in Human Genetics; regularly uptdated; Wiley & Sons, Inc; Editors: Nicholas C. Dracopoli, Honathan L. Haines, Bruce R. Korf, Cynthia C. Morton, Christine E. Seidman, J. G. Seigman, Douglas R. Smith.

Current Protocols in Protein Science; regularly updated; Wiley & Sons, Inc; Editors: John E. Coligan, Ben M. Dunn, Hidde L. Ploegh, David W. Speicher, Paul T. Wingfield.

Molecular Biology of the Cell; third edition; Alberts, B., Bray, D., Lewis, J., Raff, M., Roberts, K., Watson, J. D.; Garland Publishing, Inc. New York & London, 1994;

Short Protocols in Molecular Biology, 5th edition, by Frederick M. Ansubel (Editor), Roger Brent (Editor), Robert E. Kingston (Editor), David D. Moore (Editor), J. G. Seidman (Editor), John A. Smith (Editor), Kevin Struhl (Editor), October 2002, John Wiley & Sons, Inc., New York”

Gene targeting: A Practical Approach, 2^{nd} Ed., Joyner A L, ed. 2000. IRL Press at Oxford University Press, New York;

1. A method of identifying a substance altering glucose uptake of a cell comprising
(a) contacting a test system comprising AKT substrate 160kDa-like protein (AS160-like protein) or functional variant thereof with a test substance, and
(b) identifying a test substance as a substance altering glucose uptake and/or GLUT4 translocation to the plasma membrane of a cell by detecting a signal indicative for altered glucose uptake of a cell.

2. The method of claim 1, wherein glucose uptake of the cell is increased or decreased, preferably increased.

3. The method of claim 1, wherein the substance alters glucose uptake and/or GLUT4 translocation to the plasma membrane in at least one, two or three insulin-sensitive tissues.

4. The method of claim 3, wherein the insulin-sensitive tissue is adipose tissue, skeletal muscle and/or liver.

5. The method of claim 1, wherein the substance alters glucose uptake and/or GLUT4 translocation to the plasma membrane of a hepatocyte, an adipocyte and/or a skeletal muscle cell.

6. The method of claim 1, wherein the AS160-like protein is mammalian and preferably human AS160-like protein.

7. The method of claim 1, wherein the AS 160-like protein is isoform 2 of AS160.

8. The method of claim 1, wherein the AS 160-like protein is isoform 3 of AS160.

9. The method of claim 1, wherein the AS160-like protein comprises or consists of the sequence encoded by SEQ ID NO: 1 or 22.

10. The method of claim 1, wherein the test system is in a cell.

11. The method of claim 10, wherein the cell is a skeletal muscle cell, an adipocyte and/or hepatocyte, particularly a skeletal muscle cell.

12. The method of claim 10, wherein the cell is a cell from a cell line.

13. The method of claim 1, wherein the detectable signal is the amount of AS160-like protein expressed in a cell, phosphorylated AKT, phosphorylated AS160-like protein, GLUT4 translocation to the plasma membrane, GLUT4 distribution in a cell or glucose uptake by a cell.

14. The method of claim 1, wherein the test compound is provided in the form of a chemical compound library.

15. The method of claim 1, wherein the method is carried out in a robotics system.

16. The method according to claim 1, wherein the method is a method of high-through put screening.

17. A test system for the identification of a substance for improving glucose uptake and/or GLUT4 translocation to the plasma membrane of a cell, the test system comprising a gene coding for the AKT substrate 160 kDa-like protein (AS160-like protein) or functional variant thereof; and an inducible promoter providing for controllable expression of the gene, wherein the activation of AS160-like protein or functional variant thereof effects a detectable signal.

18. The test system of claim 17, wherein the test system is located in a cell, particularly a genetically engineered cell.

19. The test system of claim 18, wherein the gene or the promoter is introduced into the genetically engineered cell.

20. The test system of claim 17, wherein the inducible promoter is a tetracycline-inducible promoter.

21. (canceled)

22. A method of identifying a substance altering glucose uptake or GLUT4 translocation to the plasma membrane of a cell, the method comprising:
(a) applying a test substance to the test system of claim 17; and
(b) identifying the test substance as a substance altering glucose uptake or GLUT4 translocation to the plasma membrane of a cell by detecting a signal indicative for altered glucose uptake of a cell.

23-27. (canceled)

28. A polypeptide consisting or essentially consisting of the amino acid sequence according to SEQ ID NO: 3 or 23 or being encoded by a sequence according to SEQ ID NO: 1 or 22.

29. A polynucleotide consisting of or essentially consisting of polynucleotide sequence according to SEQ ID NO: 1 or 22 or encoding a polypeptide according to SEQ ID NO: 3 or 23.

30. An antibody specifically binding to a polypeptide according to claim 28.

31. A cell heterologously expressing the polypeptide according to claim 28.

32. A cell stably or transiently transfected with a polynucleotide according to claim 29.

33. A siRNA able to negatively interfere with expression and/or activity of AS160-like protein.

34. (canceled)

35. A siRNA according to claim 33, wherein the AS160-like protein is isoform 2 or 3 of AS160 protein.


SEQUENCE LISTING (23 sequences)

SEQ ID NO: 1 - DNA, 3707 bp, Homo sapiens
atggagccgcccagctgcattcaggatgagccgttcccgcaccccctggagcccgagccgggcgtctcagctcagcccggccccgggaagccaagcgataagcggttccggctgtggtacgttggggggtcgtgcctggaccacaggaccacgctgcctatgctgccctggctcatggccgagatccgcaggcgcagccagaagcccgaggcgggcggctgcggggcgccggcggcccgagaggtgatcctggtgctcagcgcgcccttcctgcgttgcgtccccgcgccgggcgctggggcctcggggggcactagtccgtcggccacgcagcccaacccggcggtattcatcttcgagcacaaggcgcagcatatctcgcgcttcatccacaacagccacgacctcacctactttgcctacctgatcaaggcgcagcccgacgaccccgagtcgcagatggcctgccacgttttccgcgccacagaccccagccaggttcctgatgttattagcagcataaggcaattatctaaagcggccatgaaagaggatgccaaacccagcaaagataatgaggacgccttttacaactctcagaagtttgaagtcctgtactgtggaaaggtgaccgtgacccacaagaaggccccctcaagcctcatcgatgactgcatggagaagttcagcctgcacgaacagcagcgcctgaagatccaaggcgagcagcgcggtccggacccaggagaggacctggctgacttggaggtggtggtgcccgggtcccccggagactgcctgccggaggaggctgacggcaccgacacccaccttggcttacctgccggggccagccagcctgccctgaccagctctcgggtctgcttccctgagcggattttggaagattctggctttgatgagcagcaggagtttcggtctcggtgcagcagtgtcaccggcgtgcaacggagagttcacgagggcagccagaaatcccagccgcgacggagacacgcgagcgcacccagtcacgtccagccctcggactcggagaagaacaggaccatgctcttccaggttgggcgatttgagattaaccttatcagtccagacactaaatcagttgtgctagaaaagaattttaaagatatctcctcttgttctcagggtataaagcatgtggatcactttggctttatctgccgggagtctccagagcctggacttagccagtatatttgttatgtattccagtgtgccagcgaatctctggttgatgaggtaatgctgactctgaaacaggccttcagtacggcggctgccctgcagagtgccaagacgcagattaaactgtgtgaggcctgcccgatgcactctttgcataagctctgtgaaaggattgaaggtctctacccaccaagagccaagctggtgatacagaggcatctctcatcactgacagataatgagcaagctgacatctttgaaagagttcagaaaatgaagccagtcagtgaccaggaagaaaatgaacttgtgattttacacctgaggcagctgtgtgaagccaagcagaagacacacgtgcacatcggggaaggcccttctactatttcaaatagtacaatcccagaaaatgcaacaagcagtggaaggttcaaacttgacattctgaaaaataaagctaagagatccttaactagctccctggaaaatatcttctcaaggggagctaacagaatgagaggtcggcttggaagtgtggacagttttgaacggtccaacagtcttgcttcagagaaggactactcaccaggggattctccaccagggacaccgccagcgtccccaccgtcctcagcttggcaaacgtttcccgaagaggattccgactccccgcagtttcgaagacgggcacacacgttcagccacccaccttcaagcacaaagagaaagctgaatttgcaggatgggagggctcagggtgtgcgttcccctctgctgaggcagagctccagtgaacagtgcagtgatggagaagggagaaaaaggacctcatctacctgcagcaatgagtccctaagtgtgggaggaacctctgtcactcctcgccggatctcctggcggcagcgcattttcctcagggttgcttctcccatgaacaaatctccctcagcaatgcaacagcaagatggattggacaggaacgagctgctgccactgtcccccctctctccaaccatggaggaggaaccgctggttatattcctgtctggggaggatgacccagaaaagattgaagaaagaaagaaatcaaaagaactgaggagcttgtggagaaaagctatacaccaacaaatcttgttacttcgaatggaaaaagaaaaccagaaacttgaaggagcaagcagagatgaactccagtccagaaaagttaaattagactatgaagaagttggtgcatgtcagaaagaggtcttaataacttgggataagaagttgttaaactgcagagctaaaatcagatgtgatatggaagatattcatactcttcttaaagaaggagttcccaaaagtcgacgaggagaaatttggcagtttctggctttacagtaccgactcagacacagattgcctaataaacaacagcctcctgacatatcctataaggaacttttgaagcagctcactgctcagcagcatgcgattctcgtggatttaggaaggacgtttcctactcacccttacttttcagtacagcttgggccaggacagctgtcactgtttaacctcctgaaagcctattctttgctggacaaagaagtgggatactgtcaggggatcagctttgtggctggagtcctgcttctgcacatgagtgaagagcaagcctttgaaatgctgaaattcctcatgtatgacctcggcttccgcaagcagtacagacctgacatgatgtcgctgcagattcaaatgtaccagctgtccaggctccttcatgactatcacagagatctctacaatcaccttgaagaaaatgaaatcagccccagtctttatgctgccccctggttcctcacattgtttgcctctcagttttcattaggatttgtagccagagtttttgatattatttttcttcagggaactgaagttatattcaaggttgcactcagcctactgagcagccaagagacacttataatggaatgtgagagctttgaaaatattgttgagtttcttaaaaacacgctacctgatatgaatacctctgaaatggaaaaaattattacccaggtttttgagatggatatttctaagcagttgcatgcctatgaggtggaatatcatgtgctacaggatgagcttcaggaatcttcatattcctgtgaggatagtgaaactttggagaagctggagagggccaatagccaactgaaaagacaaaacatggacctcctagaaaaattacaggtagctcatactaaaatccaggccttggaatcaaacctggaaaatcttttgacgagagagaccaaaatgaagtctttaatccggaccctggaacaagaaaaaatggcttatcaaaagacagtggagcaactccggaagctgctgcccgcggatgctctagtcaattgtgacctgttgctgagagacctaaactgcaaccctaacaacaaagccaagataggaaataagcc

SEQ ID NO: 2 - DNA, 5818 bp, Artificial
  Vector pIRES-puro2/TetR
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcgatatctgcggcctagctagcgtttaaacttaagcttaccatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaagcggccgcatagataactgatccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagtgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - protein, 1236 aa, Homo sapiens
MEPPSCIQDEPFPHPLEPEPGVSAQPGPGKPSDKRFRLWYVGGSCLDHRTTLPMLPWLMAEIRRRSQKPEAGGCGAPAAREVILVLSAPFLRCVPAPGAGASGGTSPSATQPNPAVFIFEHKAQHISRFIHNSHDLTYFAYLIKAQPDDPESQMACHVFRATDPSQVPDVISSIRQLSKAAMKEDAKPSKDNEDAFYNSQKFEVLYCGKVTVTHKKAPSSLIDDCMEKFSLHEQQRLKIQGEQRGPDPGEDLADLEVVVPGSPGDCLPEEADGTDTHLGLPAGASQPALTSSRVCFPERILEDSGFDEQQEFRSRCSSVTGVQRRVHEGSQKSQPRRRHASAPSHVQPSDSEKNRTMLFQVGRFEINLISPDTKSVVLEKNFKDISSCSQGIKHVDHFGFICRESPEPGLSQYICYVFQCASESLVDEVMLTLKQAFSTAAALQSAKTQIKLCEACPMHSLHKLCERIEGLYPPRAKLVIQRHLSSLTDNEQADIFERVQKMKPVSDQEENELVILHLRQLCEAKQKTHVHIGEGPSTISNSTIPENATSSGRFKLDILKNKAKRSLTSSLENIFSRGANRMRGRLGSVDSFERSNSLASEKDYSPGDSPPGTPPASPPSSAWQTFPEEDSDSPQFRRRAHTFSHPPSSTKRKLNLQDGRAQGVRSPLLRQSSSEQCSDGEGRKRTSSTCSNESLSVGGTSVTPRRISWRQRIFLRVASPMNKSPSAMQQQDGLDRNELLPLSPLSPTMEEEPLVIFLSGEDDPEKIEERKKSKELRSLWRKAIHQQILLLRMEKENQKLEGASRDELQSRKVKLDYEEVGACQKEVLITWDKKLLNCRAKIRCDMEDIHTLLKEGVPKSRRGEIWQFLALQYRLRHRLPNKQQPPDISYKELLKQLTAQQHAILVDLGRTFPTHPYFSVQLGPGQLSLFNLLKAYSLLDKEVGYCQGISFVAGVLLLHMSEEQAFEMLKFLMYDLGFRKQYRPDMMSLQIQMYQLSRLLHDYHRDLYNHLEENEISPSLYAAPWFLTLFASQFSLGFVARVFDIIFLQGTEVIFKVALSLLSSQETLIMECESFENIVEFLKNTLPDMNTSEMEKIITQVFEMDISKQLHAYEVEYHVLQDELQESSYSCEDSETLEKLERANSQLKRQNMDLLEKLQVAHTKIQALESNLENLLTRETKMKSLIRTLEQEKMAYQKTVEQLRKLLPADALVNCDLLLRDLNCNPNNKAKIGNKP

SEQ ID NO: 4 - DNA, 28 bp, Artificial
  Primer/Probe
catactcttcttaaagaaggagttccca

SEQ ID NO: 5 - DNA, 26 bp, Artificial
  Primer/Probe
ctgtgtctgagtcggtactgtaaagc

SEQ ID NO: 6 - DNA, 30 bp, Artificial
  Primer/Probe
cagaaactgccaaatttctcctcgtcgact

SEQ ID NO: 7 - DNA, 18 bp, Artificial
  Primer/Probe
gcgttcccctctgctgag

SEQ ID NO: 8 - DNA, 23 bp, Artificial
  Primer/Probe
actcattgctgcaggtagatgag

SEQ ID NO: 9 - DNA, 30 bp, Artificial
  Primer/Probe
ttctccatcactgcactgttcactggagct

SEQ ID NO: 10 - DNA, 23 bp, Artificial
  Primer/Probe
acagcggaagtggtattgtacgt

SEQ ID NO: 11 - DNA, 19 bp, Artificial
  Primer/Probe
ggcactgtggttcctgcat

SEQ ID NO: 12 - DNA, 22 bp, Artificial
  Primer/Probe
caggcaccgccagccactgtct

SEQ ID NO: 13 - DNA, 5055 bp, Homo sapiens
gcggccgcggggaccctcggcgtggtcctctgaccctgcaaacccgcgacggaggaaggggaggtcctgcccgaggcgccagcccaaggaggaggatgcccatttaacccgccctcgcctgccgggcgcttgcctcggtgcccgccgccggagcctccgagccgcgcccgtggaagtgctgcatggggcagggctgctgaagcgcggagttcggggtcgcgccgctcccaggcaggcgcgggagcccggtgcggcagttggcacagtttcggcggcgccttctgcgcgggagtggggggcgcggtgcgcccggccggcctccgcggtgccctggtgaggcgagagttatggagccgcccagctgcattcaggatgagccgttcccgcaccccctggagcccgagccgggcgtctcagctcagcccggccccgggaagccaagcgataagcggttccggctgtggtacgttggggggtcgtgcctggaccacaggaccacgctgcctatgctgccctggctcatggccgagatccgcaggcgcagccagaagcccgaggcgggcggctgcggggcgccggcggcccgagaggtgatcctggtgctcagcgcgcccttcctgcgttgcgtccccgcgccgggcgctggggcctcggggggcactagtccgtcggccacgcagcccaacccggcggtattcatcttcgagcacaaggcgcagcatatctcgcgcttcatccacaacagccacgacctcacctactttgcctacctgatcaaggcgcagcccgacgaccccgagtcgcagatggcctgccacgttttccgcgccacagaccccagccaggttcctgatgttattagcagcataaggcaattatctaaagcggccatgaaagaggatgccaaacccagcaaagataatgaggacgccttttacaactctcagaagtttgaagtcctgtactgtggaaaggtgaccgtgacccacaagaaggccccctcaagcctcatcgatgactgcatggagaagttcagcctgcacgaacagcagcgcctgaagatccaaggcgagcagcgcggtccggacccaggagaggacctggctgacttggaggtggtggtgcccgggtcccccggagactgcctgccggaggaggctgacggcaccgacacccaccttggcttacctgccggggccagccagcctgccctgaccagctctcgggtctgcttccctgagcggattttggaagattctggctttgatgagcagcaggagtttcggtctcggtgcagcagtgtcaccggcgtgcaacggagagttcacgagggcagccagaaatcccagccgcgacggagacacgcgagcgcacccagtcacgtccagccctcggactcggagaagaacaggaccatgctcttccaggttgggcgatttgagattaaccttatcagtccagacactaaatcagttgtgctagaaaagaattttaaagatatctcctcttgttctcagggtataaagcatgtggatcactttggctttatctgccgggagtctccagagcctggacttagccagtatatttgttatgtattccagtgtgccagcgaatctctggttgatgaggtaatgctgactctgaaacaggccttcagtacggcggctgccctgcagagtgccaagacgcagattaaactgtgtgaggcctgcccgatgcactctttgcataagctctgtgaaaggattgaaggtctctacccaccaagagccaagctggtgatacagaggcatctctcatcactgacagataatgagcaagctgacatctttgaaagagttcagaaaatgaagccagtcagtgaccaggaagaaaatgaacttgtgattttacacctgaggcagctgtgtgaagccaagcagaagacacacgtgcacatcggggaaggcccttctactatttcaaatagtacaatcccagaaaatgcaacaagcagtggaaggttcaaacttgacattctgaaaaataaagctaagagatccttaactagctccctggaaaatatcttctcaaggggagctaacagaatgagaggtcggcttggaagtgtggacagttttgaacggtccaacagtcttgcttcagagaaggactactcaccaggggattctccaccagggacaccgccagcgtccccaccgtcctcagcttggcaaacgtttcccgaagaggattccgactccccgcagtttcgaagacgggcacacacgttcagccacccaccttcaagcacaaagagaaagctgaatttgcaggatgggagggctcagggtgtgcgttcccctctgctgaggcagagctccagtgaacagtgcagtgatggagaagggagaaaaaggacctcatctacctgcagcaatgagtccctaagtgtgggaggaacctctgtcactcctcgccggatctcctggcggcagcgcattttcctcagggttgcttctcccatgaacaaatctccctcagcaatgcaacagcaagatggattggacaggaacgagctgctgccactgtcccccctctctccaaccatggaggaggaaccgctggttatattcctgtctggggaggatgacccagaaaagattgaagaaagaaagaaatcaaaagaactgaggagcttgtggagaaaagctatacaccaacaaatcttgttacttcgaatggaaaaagaaaaccagaaacttgaaggagcaagcagagatgaactccagtccagaaaagttaaattagactatgaagaagttggtgcatgtcagaaagaggtcttaataacttgggataagaagttgttaaactgcagagctaaaatcagatgtgatatggaagatattcatactcttcttaaagaaggagttcccaaaagtcgacgaggagaaatttggcagtttctggctttacagtaccgactcagacacagattgcctaataaacaacagcctcctgacatatcctataaggaacttttgaagcagctcactgctcagcagcatgcgattctcgtggatttaggaaggacgtttcctactcacccttacttttcagtacagcttgggccaggacagctgtcactgtttaacctcctgaaagcctattctttgctggacaaagaagtgggatactgtcaggggatcagctttgtggctggagtcctgcttctgcacatgagtgaagagcaagcctttgaaatgctgaaattcctcatgtatgacctcggcttccgcaagcagtacagacctgacatgatgtcgctgcagattcaaatgtaccagctgtccaggctccttcatgactatcacagagatctctacaatcaccttgaagaaaatgaaatcagccccagtctttatgctgccccctggttcctcacattgtttgcctctcagttttcattaggatttgtagccagagtttttgatattatttttcttcagggaactgaagttatattcaaggttgcactcagcctactgagcagccaagagacacttataatggaatgtgagagctttgaaaatattgttgagtttcttaaaaacacgctacctgatatgaatacctctgaaatggaaaaaattattacccaggtttttgagatggatatttctaagcagttgcatgcctatgaggtggaatatcatgtgctacaggatgagcttcaggaatcttcatattcctgtgaggatagtgaaactttggagaagctggagagggccaatagccaactgaaaagacaaaacatggacctcctagaaaaattacaggtagctcatactaaaatccaggccttggaatcaaacctggaaaatcttttgacgagagagaccaaaatgaagtctttaatccggaccctggaacaagaaaaaatggcttatcaaaagacagtggagcaactccggaagctgctgcccgcggatgctctagtcaattgtgacctgttgctgagagacctaaactgcaaccctaacaacaaagccaagataggaaataagccccaggtttttgagatggatatttctaagcagttgcatgcctatgaggtggaatatcatgtgctacaggatgagcttcaggaatcttcatattcctgtgaggatagtgaaactttggagaagctggagagggccaatagccaactgaaaagacaaaacatggacctcctagaaaaattacaggtagctcatactaaaatccaggccttggaatcaaacctggaaaatcttttgacgagagagaccaaaatgaagtctttaatccggaccctggaacaagaaaaaatggcttatcaaaagacagtggagcaactccggaagctgctgcccgcggatgctctagtcaattgtgacctgttgctgagagacctaaactgcaaccctaacaacaaagccaagataggaaataagccataattgaagaggcacggcctcagcagaaagtgctccttagaatactacagagaggaagagcctgcatgtcgctggcccaaggctggaccctgaagctgatggaaccacctaatactggtgctgagcgcctagtcacagcaggtggacctcgtgctcatcagagcatgccaatcctaagccattggacatatgtagactggtttttgttgttgctatgtacatataaatatatatataaaatgaacatagttcatgctttcagataaaatgagtagatgtatatttagattaatttttttagtcagaacttcatgaaatccacaccaaaggaaaggtaaactgaaatttcccttggacatatgtgaaatctttttgtctttatagtgaaacaaagccagagcatctttgtatattgcaatatacttgaaaaaaatgaatgtatttttttctccaaagaacagcatgtttcactcaatggtgaaaaggtggaaacatttatgtaactttatgtgtatctgtcttgatatctactgacattgtctatatgaggaaaatgattactggtcatgctcctgtgagttttttggga

SEQ ID NO: 14 - DNA, 21 bp, Artificial
  Primer/Probe
ggaggaggatgcccatttaac

SEQ ID NO: 15 - DNA, 23 bp, Artificial
  Primer/Probe
tctaaggagcactttctgctgag

SEQ ID NO: 16 - DNA, 28 bp, Artificial
  Primer/Probe
agcttttaccagaattcaggtagactgt

SEQ ID NO: 17 - DNA, 22 bp, Artificial
  Primer/Probe
tgctgcaggtagatgaggtcct

SEQ ID NO: 18 - DNA, 22 bp, Artificial
  Primer/Probe
cttctccatcactgatttcatt

SEQ ID NO: 19 - DNA, 26 bp, Artificial
  Primer/Probe
attcaggtagactgtccccacagtat

SEQ ID NO: 20 - DNA, 25 bp, Artificial
  Primer/Probe
ccttctccatcacttgattctgaag

SEQ ID NO: 21 - DNA, 21 bp, Artificial
  Primer/Probe
atgaaatcagacaagacactg

SEQ ID NO: 22 - DNA, 3881 bp, Homo sapiens
atggagccgcccagctgcattcaggatgagccgttcccgcaccccctggagcccgagccgggcgtctcagctcagcccggccccgggaagccaagcgataagcggttccggctgtggtacgttggggggtcgtgcctggaccacaggaccacgctgcctatgctgccctggctcatggccgagatccgcaggcgcagccagaagcccgaggcgggcggctgcggggcgccggcggcccgagaggtgatcctggtgctcagcgcgcccttcctgcgttgcgtccccgcgccgggcgctggggcctcggggggcactagtccgtcggccacgcagcccaacccggcggtattcatcttcgagcacaaggcgcagcatatctcgcgcttcatccacaacagccacgacctcacctactttgcctacctgatcaaggcgcagcccgacgaccccgagtcgcagatggcctgccacgttttccgcgccacagaccccagccaggttcctgatgttattagcagcataaggcaattatctaaagcggccatgaaagaggatgccaaacccagcaaagataatgaggacgccttttacaactctcagaagttcgaagtcctgtactgtggaaaggtgaccgtgacccacaagaaggccccctcaagcctcatcgatgactgcatggagaagttcagcctgcacgaacagcagcgcctgaagatccaaggggagcagcgcggtccggacccaggagaggacctggctgacttggaggtggtggtgcccgggtcccccggagactgcctgccggaggaggctgacggcaccgacacccaccttggcttacctgccggggccagccagcctgccctgaccagctctcgggtctgcttccctgagcggattttggaagattctggctttgatgagcagcaggagtttcggtctcggtgcagcagtgtcaccggcgtgcaacggagagttcacgagggcagccagaaatcccagccgcgacggagacacgcgagcgcacccagtcacgtccagccctcggactcggagaagaacaggaccatgctcttccaggttgggcgatttgagattaaccttatcagtccagacactaaatcagttgtgctagaaaagaattttaaagatatctcctcttgttctcagggtataaagcatgtggatcactttggctttatctgccgggagtctccagagcctggacttagccagtatatttgttatgtattccagtgtgccagcgaatctctggttgatgaggtaatgctgactctgaaacaggccttcagtacggcggctgccctgcagagtgccaagacgcagattaaactgtgtgaggcctgcccgatgcactctttgcataagctctgtgaaaggattgaaggtctctacccaccaagagccaagctggtgatacagaggcatctctcatcactgacagataatgagcaagctgacatctttgaaagagttcagaaaatgaagccagtcagtgaccaggaagaaaatgaacttgtgattttacacctgaggcagctgtgtgaagccaagcagaagacacacgtgcacatcggggaaggcccttctactatttcaaatagtacaatcccagaaaatgcaacaagcagtggaaggttcaaacttgacattctgaaaaataaagctaagagatccttaactagctccctggaaaatatcttctcaaggggagctaacagaatgagaggtcggcttggaagtgtggacagttttgaacggtccaacagtcttgcttcagagaaggactactcaccaggggattctccaccagggacaccgccagcgtccccaccgtcctcagcttggcaaacgtttcccgaagaggattccgactccccgcagtttcgaagacgggcacacacgttcagccacccaccttcaagcacaaagagaaagctgaatttgcaggatgggagggctcagggtgtgcgttcccctctgctgaggcagagctccagtgaacagtgcagcaatctttcgtcagttcgacgcatgtacaaggagagtaattcttcctccagtcttccaagtcttcacacttccttctctgccccttccttcactgccccctctttcctgaaaagcttttaccagaattcaggtagactgtccccacagtatgaaaatgaaatcagactgatggagaagggagaaaaaggacctcatctacctgcagcaatgagtccctaagtgtgggaggaacctctgtcactcctcgccggatctcctggcggcagcgcattttcctcagggttgcttctcccatgaacaaatctccctcagcaatgcaacagcaagatggattggacaggaacgagctgctgccactgtcccccctctctccaaccatggaggaggaaccgctggttgtattcctgtctggggaggatgacccagaaaagattgaagaaagaaagaaatcaaaagaactgaggagcttgtggagaaaagctatacaccaacaaatcttgttacttcgaatggaaaaagaaaaccagaaacttgaagcaagcagagatgaactccagtccagaaaagttaaattagactatgaagaagttggtgcatgtcagaaagaggtcttaataacttgggataagaagttgttaaactgcagagctaaaatcagatgtgatatggaagatattcatactcttcttaaagaaggagttcccaaaagtcgacgaggagaaatttggcagtttctggctttacagtaccgactcagacacagattgcctaataaacaacagcctcctgacatatcctataaggaacttttgaagcagctcactgctcagcagcatgcgattctcgtggatttaggaaggacgtttcctactcacccttacttttcagtacagcttgggccaggacagctgtcactgtttaacctcctgaaagcctattctttgctggacaaagaagtgggatactgtcaggggatcagctttgtggctggagtcctgcttctgcacatgagtgaagagcaagcctttgaaatgctgaaattcctcatgtatgacctcggcttccgcaagcagtacagacctgacatgatgtcgctgcagattcaaatgtaccagctgtccaggctccttcatgactatcacagagatctctacaatcaccttgaagaaaatgaaatcagccccagtctttatgctgccccctggttcctcacattgtttgcctctcagttttcattaggatttgtagccagagtttttgatattatttttcttcagggaactgaagttatattcaaggttgcactcagcctactgagcagccaagagacacttataatggaatgtgagagctttgaaaatattgttgagtttcttaaaaacacgctacctgatatgaatacctctgaaatggaaaaaattattacccaggtttttgagatggatatttctaagcagttgcatgcctatgaggtggaatatcatgtgctacaggatgagcttcaggaatcttcatattcctgtgaggatagtgaaactttggagaagctggagagggccaatagccaactgaaaagacaaaacatggacctcctagaaaaattacaggtagctcatactaaaatccaggccttggaatcaaacctggaaaatcttttgacgagagagaccaaaatgaagtctttaatccggaccctggaacaagaaaaaatggcttatcaaaagacagtggagcaactccggaagctgctgcccgcggatgctctagtcaattgtgacctgttgctgagagacctaaactgcaaccctaacaacaaagccaagataggaaataagccataattgaag

SEQ ID NO: 23 - protein, 1264 aa, Homo sapiens
MEPPSCIQDEPFPHPLEPEPGVSAQPGPGKPSDKRFRLWYVGGSCLDHRTTLPMLPWLMAEIRRRSQKPEAGGCGAPAAREVILVLSAPFLRCVPAPGAGASGGTSPSATQPNPAVFIFEHKAQHISRFIHNSHDLTYFAYLIKAQPDDPESQMACHVFRATDPSQVPDVISSIRQLSKAAMKEDAKPSKDNEDAFYNSQKFEVLYCGKVTVTHKKAPSSLIDDCMEKFSLHEQQRLKIQGEQRGPDPGEDLADLEVVVPGSPGDCLPEEADGTDTHLGLPAGASQPALTSSRVCFPERILEDSGFDEQQEFRSRCSSVTGVQRRVHEGSQKSQPRRRHASAPSHVQPSDSEKNRTMLFQVGRFEINLISPDTKSVVLEKNFKDISSCSQGIKHVDHFGFICRESPEPGLSQYICYVFQCASESLVDEVMLTLKQAFSTAAALQSAKTQIKLCEACPMHSLHKLCERIEGLYPPRAKLVIQRHLSSLTDNEQADIFERVQKMKPVSDQEENELVILHLRQLCEAKQKTHVHIGEGPSTISNSTIPENATSSGRFKLDILKNKAKRSLTSSLENIFSRGANRMRGRLGSVDSFERSNSLASEKDYSPGDSPPGTPPASPPSSAWQTFPEEDSDSPQFRRRAHTFSHPPSSTKRKLNLQDGRAQGVRSPLLRQSSSEQCSNLSSVRRMYKESNSSSSLPSLHTSFSAPSFTAPSFLKSFYQNSGRLSPQYENEIRLMEKGEKGPHLPAAMSPVWEEPLSLLAGSPGGSAFSSGLLLPTNLPQQCNSKMDWTGTSCCHCPPSLQPWRRNRWLYSCLGRMTQKRLKKERNQKNGACGEKLYTNKSCYFEWKKKTRNLKQAEMNSSPEKLNTMKKLVHVRKRSLGIRSCTAELKSDVIWKIFILFLKKEFPKVDEEKFGSFWLYSTDSDTDCLINNSLLTYPIRNFSSSLLSSMRFSWIEGRFLLTLTFQYSLGQDSCHCLTSKPILCWTKKWDTVRGSALWLESCFCTVKSKPLKCNSSCMTSASASSTDLTCRCRFKCTSCPGSFMTITEISTITLKKMKSAPVFMLPPGSSHCLPLSFHDLPEFLILFFFRELKLYSRLHSAYAAKRHLWNVRALKILLSFLKTRYLIIPLKWKKLLPRFLRWIFLSSCMPMRWNIMCYRMSFRNLHIPVRIVKLWRSWRGPIANKDKTWTSKNYRLILKSRPWNQTWKIFRERPKSLSGPWNKKKWLIKRQWSNSGSCCPRMLSIVTCCETTATLTTKPREISHN